(19) United States
(12) Patent Application Publication
(10) Pub. No.: US 2026/0081036 A1
(43) Pub. Date: Mar. 19, 2026

(54) AI-DRIVEN REAL-TIME MONITORING AND PREDICTIVE ANALYTICS SYSTEM FOR ENGINEERED T CELL THERAPIES IN CANCER MANAGEMENT

(71) Applicants: Meesue Kim, Woodinville, WA (US); Yoo Seung Kim, Bangkok (TH); Tanapattsorn Maardthpiratch, Bangkok (TH); Rawiwan Charoensub, Bangkok (TH)

(72) Inventors: Meesue Kim, Woodinville, WA (US); Yoo Seung Kim, Bangkok (TH); Tanapattsorn Maardthpiratch, Bangkok (TH); Rawiwan Charoensub, Bangkok (TH)

(73) Assignee: Meesue Kim, Woodinville, WA (US)

(21) Appl. No.: 18/884,120

(22) Filed: Sep. 13, 2024

(51) Int. Cl. G16H 50/50; G16B 5/20; G16B 40/20; G16H 10/40; G16H 10/60; G16H 20/10

(52) U.S. Cl. CPC G16H 50/50; G16B 5/20; G16B 40/20; G16H 10/40; G16H 10/60; G16H 20/10

(57) ABSTRACT

The present invention relates to a specialized AI-driven data analytics platform tailored for optimizing engineered T cell therapies in patients, particularly those undergoing treatment for cancer, autoimmune diseases, and inflammatory conditions. Unlike general-purpose AI systems, this platform integrates advanced machine learning, deep learning, and fuzzy logic algorithms to continuously analyze and prioritize real-time data from multiple sources, including patient monitoring systems, laboratory tests, imaging modalities, wearable devices, and genomic profiles. The platform is specifically designed to predict and manage adverse events unique to T cell therapies, such as Cytokine Release Syndrome (CRS) and Tumor Lysis Syndrome (TLS), offering clinicians real-time, personalized guidance that dynamically adjusts treatment protocols during and after T cell infusion. The system's adaptive learning capabilities allow it to evolve by incorporating clinical feedback and patient outcomes, continuously refining its predictive models to enhance precision and effectiveness. By providing robust support for managing complex side effects and delivering actionable recommendations, this invention marks a significant advancement in the application of AI to oncology, offering a highly specialized, innovative approach to enhancing the safety and efficacy of engineered T cell therapies.

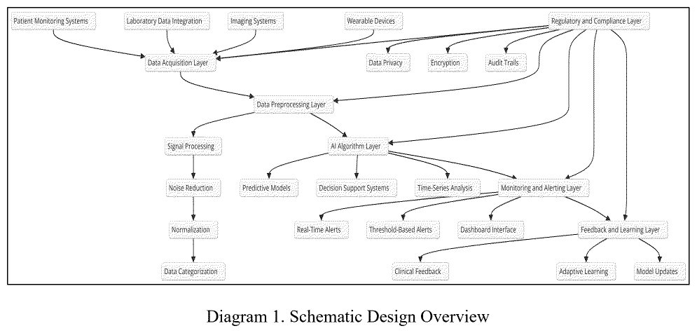

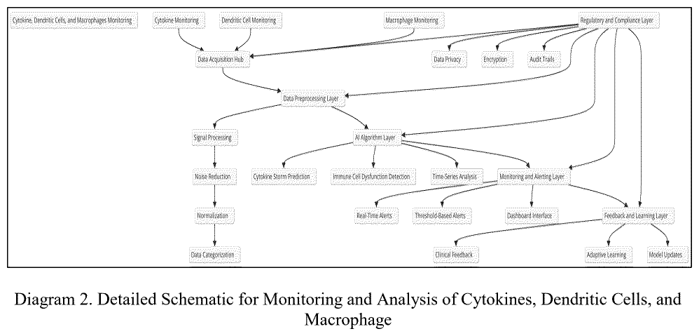

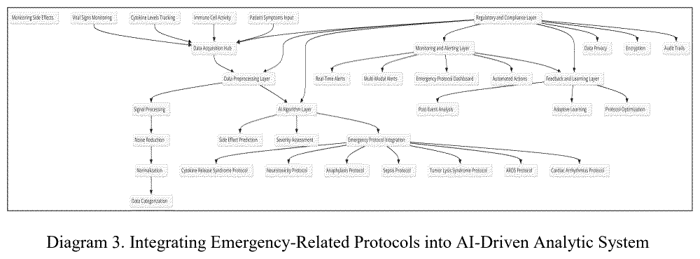

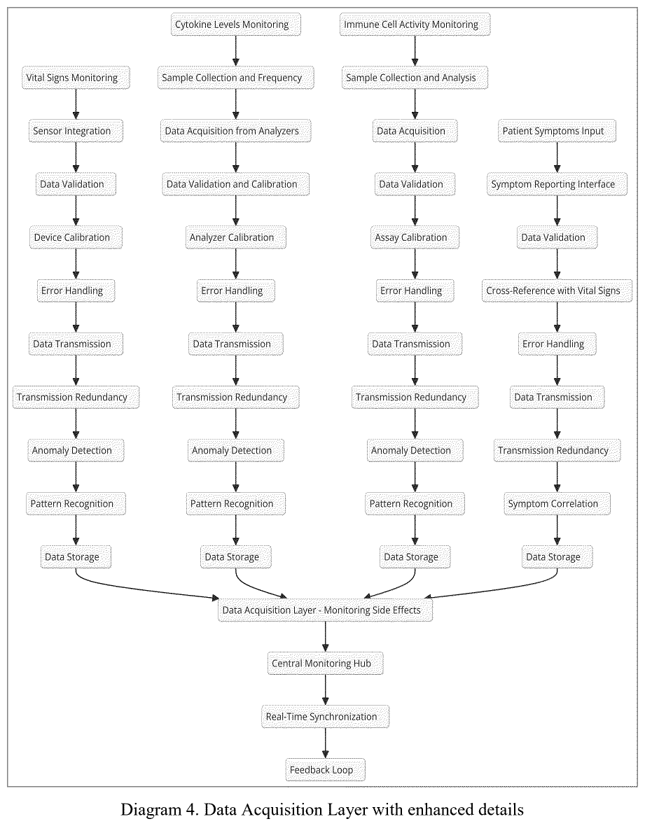

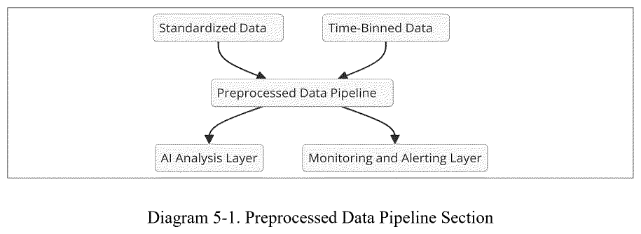

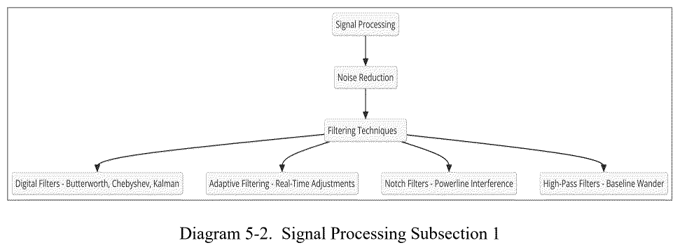

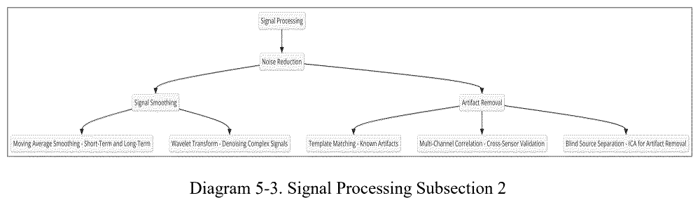

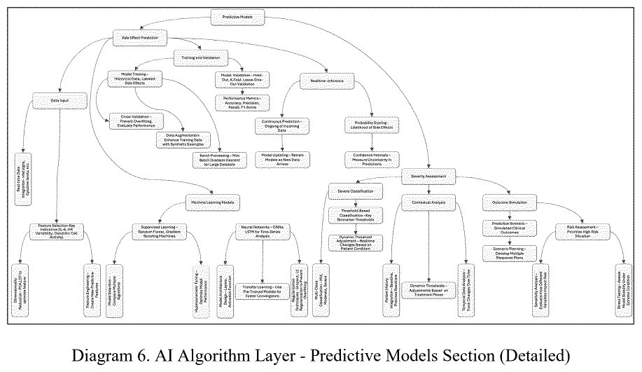

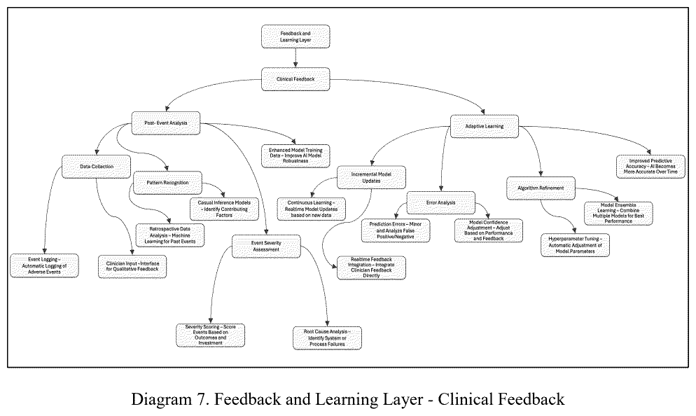

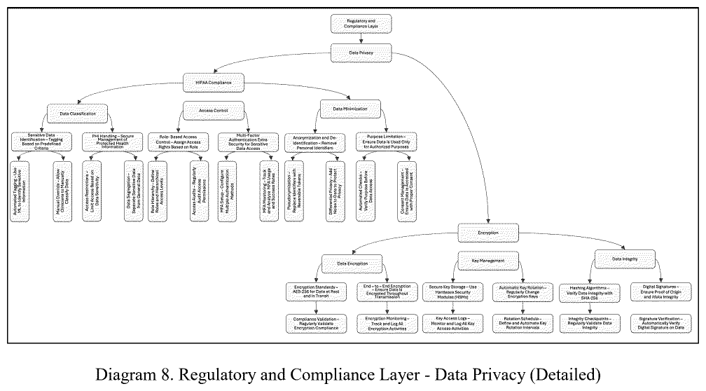

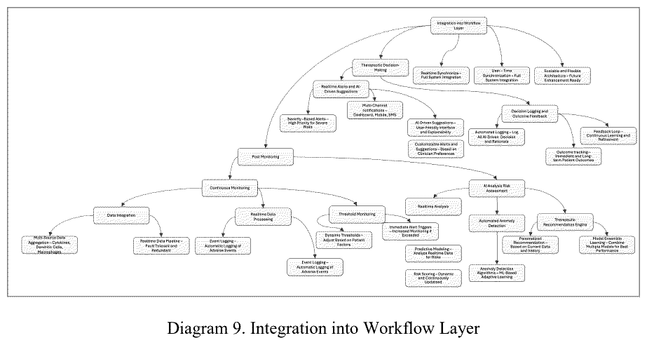

### FIELDS

The present invention pertains to the field of biotechnology and medical data analytics, specifically focusing on the integration of artificial intelligence (AI) and machine learning (ML) techniques with engineered T cell therapies for the treatment of cancer, autoimmune diseases, and infectious diseases. The invention addresses the critical challenges of real-time monitoring, predictive analytics, and personalized therapeutic recommendations in the context of advanced cellular therapies, including Chimeric Antigen Receptor (CAR) T cells, Gamma Delta T cells, and other adoptive T cell-based immunotherapies.

More specifically, this invention lies at the intersection of immunotherapy, computational biology, and clinical decision support systems (CDSS), utilizing sophisticated AI-driven platforms to optimize the management of patients undergoing T cell therapies. The invention is designed to enhance the precision and efficacy of immunotherapeutic treatments by continuously integrating and analyzing multi-modal patient data from clinical, laboratory, and molecular sources in real-time. The system facilitates the proactive management of severe and potentially life-threatening side effects, such as Cytokine Release Syndrome (CRS), Tumor Lysis Syndrome (TLS), and other immune-related adverse events, that are often associated with the infusion of engineered T cells.

The invention is also situated in the field of digital health and bioinformatics, as it leverages real-time data streams from wearable devices, medical imaging modalities, and genomic profiling tools. By utilizing advanced AI methodologies, such as deep learning, reinforcement learning, and fuzzy logic, the system provides clinicians with highly contextualized, personalized treatment strategies that dynamically adapt to the patient's evolving clinical condition. The system's ability to process large datasets and derive actionable insights from complex biological interactions distinguishes it from traditional monitoring platforms, making it a significant advancement in the management of immune-oncologyand other T cell-mediated therapies.

Furthermore, this invention is rooted in healthcare informatics, where the need for seamless integration with electronic health records (EHRs), laboratory information systems (LIS), and pharmacy management systems is paramount. The invention aims to streamline clinical workflows by automating data acquisition, analysis, and decision-making processes, reducing the cognitive and operational burden on healthcare providers. This AI-driven platform also enhances interdisciplinary collaboration by providing a comprehensive dashboard for real-time data visualization, facilitating communication between oncologists, immunologists, nurses, and pharmacists.

The scope of this invention also extends to the field of regenerative medicine and cellular therapy logistics, as it supports the management of complex logistics related to the manufacturing, quality control, and delivery of engineered T cells. This involves optimizing the cryogenic preservation and transport of cellular therapies, ensuring the safety and viability of the therapeutic cells before, during, and after infusion.

In summary, the invention operates within multiple fields, including:
1. Immunotherapy and Cellular Therapy: Encompassing T cell engineering techniques, adoptive T cell transfer, and immune modulation for cancer and autoimmune diseases.
2. Artificial Intelligence and Machine Learning in Healthcare: Utilizing AI/ML algorithms to predict adverse immune responses, optimize treatment protocols, and personalize patient care in real-time.
3. Bioinformatics and Data Analytics: Integrating patient data from diverse sources (clinical, laboratory, genomic) to generate actionable insights for therapeutic decision-making.
4. Digital Health and Remote Monitoring: Leveraging wearable technologies and remote patient monitoring systems for continuous data collection and analysis in real-world settings.
5. Healthcare Informatics: Ensuring smooth integration with existing healthcare systems such as EHRs and supporting real-time decision-making through interactive dashboards and clinical decision support tools.
6. Cryogenics and Cellular Therapy Logistics: Managing the cryopreservation, transport, and storage of engineered T cells to maintain their therapeutic efficacy across clinical and logistical settings.

By addressing the complex needs of engineered T cell therapies, this invention presents a comprehensive solution for enhancing the safety, efficacy, and scalability of these cutting-edge treatments.

### BACKGROUND OF THE INVENTION

Engineered T cell therapies, including Chimeric Antigen Receptor (CAR) T cells, Gamma-Delta T cells, and other forms of adoptive cell transfer, represent a transformative advancement in the treatment of cancer, autoimmune diseases, and inflammatory conditions. These therapies harness the patient's immune system by genetically modifying T cells to specifically target and eradicate malignant cells. Despite their groundbreaking efficacy, these therapies carry significant risks, most notably in the form of Cytokine Release Syndrome (CRS) and Tumor Lysis Syndrome (TLS). CRS, driven by the rapid activation and expansion of engineered T cells, can provoke severe and life-threatening systemic inflammatory responses. TLS, resulting from the swift destruction of tumor cells, causes dangerous metabolic imbalances as intracellular contents flood the bloodstream. The unpredictability and severity of these adverse events pose substantial challenges to clinicians, necessitating vigilant, real-time monitoring and prompt, evidence-based intervention.

Existing platforms for monitoring and managing immune responses during T cell therapies often rely on periodic data collection and static algorithms, which are reactive in nature and limited in their ability to predict and preempt adverse events. For example, platforms like IBM's Watson, while effective in general medical diagnostics and treatment recommendations, do not offer the specialized, real-time analytics necessary for the dynamic management of engineered T cell therapies. These systems typically lack the capability to integrate and analyze diverse, high-frequency data streams—such as cytokine levels, patient vitals, and genomic profiles—in real-time, which is critical for anticipating and mitigating the onset of CRS, TLS, and other severe complications.

The current gap in real-time, predictive analytics tailored specifically for T cell therapies underscores the urgent need for an advanced AI-driven platform. This platform must be capable of continuously integrating data from a multitude of sources—including patient monitoring systems, laboratory tests, imaging studies, wearable devices, and even genomic and proteomic data—to deliver dynamic, context-aware recommendations. Such a system would not only enhance the precision and timeliness of clinical decision-making but also significantly improve patient outcomes by reducing the incidence and severity of adverse events associated with engineered T cell therapies. By addressing the limitations of existing technologies, this invention represents a critical advancement in the safe and effective application of T cell therapies in clinical practice.

### BRIEF SUMMARY OF THE INVENTION

This invention pertains to a specialized AI-driven platform designed to optimize the management of engineered T cell therapies, such as CAR T cells, Gamma-Delta T cells, and other adoptive cell therapies, particularly for cancer, autoimmune diseases, and inflammatory conditions. The system is distinguished by its ability to integrate, process, and analyze real-time data from a wide array of sources, including patient monitoring systems, laboratory tests, imaging studies, wearable devices, and genomic profiles. This comprehensive data integration allows the system to continuously monitor critical immune parameters—such as cytokine levels, immune cell activity, and patient vitals—throughout the T cell therapy process. By leveraging advanced machine learning, deep learning, and fuzzy logic algorithms, the system provides real-time predictive analytics that anticipate adverse events like Cytokine Release Syndrome (CRS) and Tumor Lysis Syndrome (TLS) before they escalate, enabling preemptive intervention and reducing the risk of severe complications.

A key innovation of this system is its ability to dynamically adjust its predictive models and therapeutic recommendations based on real-time data and patient-specific factors. This includes the use of reinforcement learning, which continuously refines the system's algorithms by incorporating clinical outcomes, real-world evidence, and feedback from healthcare providers. The system's predictive models are specifically trained to recognize the unique physiological responses associated with different types of engineered T cell therapies, thereby enhancing the precision and reliability of its recommendations.

The platform also features a robust decision support module that integrates the AI-driven analytics with clinical guidelines and patient-specific data, offering personalized and actionable insights to clinicians. These insights include recommendations for adjusting T cell infusion rates, modifying pharmacological interventions, and implementing supportive care strategies, all tailored to the individual patient's needs and the current stage of their treatment.

Designed for seamless integration into existing clinical workflows, the system supports bidirectional data exchange with electronic health records (EHRs), real-time alerting, and customizable visualization dashboards that provide clinicians with an interactive, real-time view of patient data and predictive analytics. The system's adaptive learning capabilities ensure it remains relevant and effective as it continuously evolves with new data, clinical practices, and patient outcomes.

Ultimately, this invention represents a significant advancement in the application of AI to the management of engineered T cell therapies. It provides a highly specialized, reliable, and innovative approach that not only enhances the safety and efficacy of these therapies but also improves overall healthcare delivery by reducing the burden on clinicians and enabling more informed, data-driven decision-making.

### BRIEF DESCRIPTION OF THE SEVERAL VIEWS OF THE DRAWING

Diagram 1. System Architecture and Schematic Design Overview: The invention features a comprehensive AI-driven system designed to optimize the management of T cell therapies through continuous monitoring, predictive analytics, and real-time decision support. The system architecture is composed of multiple layers, each serving a critical role in ensuring the effective monitoring and management of patient data, as well as the timely identification and response to adverse events.
1) Data Acquisition Layer: The Data Acquisition Layer serves as the foundation of the system, responsible for collecting real-time data from various sources. These include patient monitoring systems that capture vital signs such as heart rate, blood pressure, and oxygen saturation (SpO2). Additionally, laboratory data is automatically integrated, providing regular updates on cytokine levels, complete blood counts (CBC), and liver and kidney functions. Imaging systems, including MRI and CT scans, are incorporated to offer insights into the anatomical status of the patient. Wearable devices further enhance data acquisition by continuously monitoring specific biomarkers like glucose levels and physical activity. The data types gathered encompass real-time data, periodic updates, and event-based alerts, ensuring a comprehensive view of the patient's condition at all times.
2) Data Preprocessing Layer: Once the data is collected, it undergoes a series of preprocessing steps to ensure accuracy and consistency. The Data Preprocessing Layer is responsible for filtering out noise from the raw signals, normalizing data from different sources to maintain consistency, and handling missing or inconsistent data points through imputation techniques. The data is then categorized based on time intervals (e.g., every 10 minutes, 30 minutes, 1 hour, 3 hours) and classified according to relevant biomarkers such as vital signs, cytokines, and blood counts. This preprocessing is crucial for preparing the data for further analysis by the AI algorithms, ensuring that the system operates on reliable and standardized information.
3) AI Algorithm Layer: The heart of the system lies in the AI Algorithm Layer, which utilizes advanced predictive models to analyze the preprocessed data. Machine learning algorithms are employed to forecast potential complications, such as the risk of cytokine storms or neurotoxicity. Deep learning models are used to identify complex patterns by combining data from multiple dimensions, such as cytokine levels and patient vitals. Time-series analysis, through models like ARIMA and LSTM, predicts trends in biomarkers over time, enabling the system to anticipate adverse events before they fully manifest. The AI also provides decision support by suggesting optimal therapeutic interventions based on the patient's current status and continuously assessing the risk of adverse events.
4) Monitoring and Alerting Layer: To ensure timely intervention, the system includes a Monitoring and Alerting Layer that provides real-time alerts and visualizations of patient data. Threshold-based alerts are triggered when any parameter crosses predefined safety thresholds, such as when SpO2 falls below 90%. Additionally, predictive alerts are generated by the AI based on its analysis of potential risks, allowing healthcare providers to respond proactively. The system also features a dashboard interface that displays real-time graphs and trends in vital signs and cytokine levels, alongside customizable alerts and interactive decision-making tools that offer various therapeutic simulations.
5) Feedback and Learning Layer: The Feedback and Learning Layer plays a pivotal role in the system's continuous improvement. Clinician input and post-event analysis are integral components that allow the system to refine its predictive models based on real-world outcomes. This adaptive learning process enables the AI to update its algorithms as new data is collected, ensuring that its recommendations and risk assessments become increasingly accurate over time. By tracking patient outcomes and incorporating clinician feedback, the system evolves to meet the dynamic needs of T cell therapy management.
6) Regulatory and Compliance Layer: Data security and compliance are paramount in the system's design. The Regulatory and Compliance Layer ensures that all patient data handling is fully compliant with regulations such as HIPAA. This includes the encryption of data during transmission and storage, as well as maintaining detailed audit trails of all AI-driven decisions and actions for regulatory review. The system prioritizes transparency by providing clear reasoning for each AI-driven recommendation, ensuring that all interventions can be reviewed and validated by human experts.

Integration and Workflow: The system is designed to integrate seamlessly into existing clinical workflows, from patient admission to post-treatment monitoring. Upon patient admission, baseline data is collected, and the AI system is initialized based on the patient's specific conditions and medical history. Continuous monitoring allows the AI to update its recommendations and alerts in real-time as new data is received. Healthcare providers can choose to follow or override AI recommendations, with all decisions logged for future reference. The outcomes of these interventions are fed back into the system, enabling it to learn and adapt, thus improving its decision-making processes over time.

Diagram 2. System Architecture and Monitoring Overview: The system is designed to provide comprehensive monitoring and analysis of cytokines, dendritic cells, and macrophages following the infusion of cell therapies. The architecture is structured to categorize and process various biomarkers based on their significance in real-time monitoring and the frequency with which they need to be observed. The system employs a multi-layered approach to ensure that critical changes in patient physiology are detected and managed promptly.
1) Monitoring and Categorization of Biomarkers Post-Cell Therapy Infusion: The system categorizes biomarkers into different monitoring frequencies based on their dynamics, the expected speed of change in response to therapy, and their impact on patient outcomes. For example, vital signs such as heart rate, blood pressure, oxygen saturation, respiratory rate, and electrocardiogram changes are monitored every 10 minutes. These indicators are highly dynamic and can change rapidly, requiring immediate intervention if abnormalities are detected. Cytokine levels, lactate, blood glucose, and serum electrolytes are monitored every 30 minutes due to their critical role in detecting systemic changes like cytokine release syndrome and metabolic imbalances.
2) Data Acquisition and Preprocessing: The Data Acquisition Layer collects real-time data from multiple sources, including blood samples for cytokine analysis, tissue samples for dendritic cell monitoring, and macrophage subtype analysis. Automated cytokine analyzers, flow cytometry, and immunohistochemistry are among the key technologies used. The Data Preprocessing Layer then processes this data, applying noise reduction techniques, normalization, and categorization to ensure accurate and standardized information for analysis. Cytokines are categorized into pro-inflammatory and anti-inflammatory groups, while immune cells are categorized by maturation status and polarization.
3) AI Algorithm and Predictive Analytics: The AI Algorithm Layer employs machine learning models, such as random forests and gradient boosting, to predict the risk of cytokine storms based on trends in cytokine levels and their correlation with vital signs. Deep learning models analyze dendritic cell and macrophage activity to detect early signs of dysfunction. Time-series analysis using models like LSTM predicts future cytokine levels and immune cell function. Decision support systems provide therapeutic recommendations, such as adjusting corticosteroid levels or administering specific drugs, based on real-time data and risk stratification.
4) Monitoring, Alerts, and Feedback: The Monitoring and Alerting Layer provides real-time alerts when cytokines exceed critical thresholds, or when abnormal dendritic cell or macrophage activity is detected. A dashboard interface visualizes trends in cytokine levels, dendritic cell counts, and macrophage polarization states, offering interactive decision-making tools for clinicians. The Feedback and Learning Layer continuously updates the AI models based on clinical feedback and outcomes, ensuring that the system evolves and improves its predictive accuracy over time.
5) Regulatory Compliance and Integration: To ensure data privacy and compliance with regulations such as HIPAA, the system employs encryption and maintains detailed audit trails of all AI-driven decisions. The integration into clinical workflows is seamless, with continuous monitoring and real-time analysis allowing clinicians to receive timely alerts and make informed therapeutic decisions. All decisions are logged, and outcomes feed back into the AI system for ongoing learning and improvement.

Diagram 3. System Overview and Emergency Protocol Integration: The system is designed to provide comprehensive monitoring and real-time response capabilities for managing adverse events associated with engineered T cell therapies. It integrates various layers of data acquisition, preprocessing, predictive analytics, and emergency response protocols into a unified AI-driven platform. This integration ensures that not only are potential side effects monitored and predicted, but appropriate interventions are also recommended or automatically initiated to mitigate risks.
1) Data Acquisition and Monitoring Layer: At the core of the system is the Data Acquisition Layer, which continuously monitors vital signs, including heart rate, blood pressure, respiratory rate, and oxygen saturation, to detect early signs of adverse reactions. In addition to these vital signs, the system tracks key cytokine levels, such as IL-6 and TNF-α, which are critical indicators of potential cytokine storms. The activity of immune cells, particularly dendritic cells, and macrophages is also monitored to identify immune dysfunctions. Manual inputs from healthcare providers regarding patient symptoms like fever, confusion, or difficulty breathing are incorporated to provide a comprehensive picture of the patient's condition.
2) Data Preprocessing and Side Effect Detection: Once the data is collected, it undergoes a series of preprocessing steps to ensure accuracy and reliability. This includes noise reduction to clean up raw signals, normalization against baseline values for each patient, and categorization of data based on potential side effects like cytokine release syndrome (CRS), neurotoxicity, and anaphylaxis. The data is organized into time-based intervals, allowing the system to track changes over time and detect patterns that may indicate the onset of adverse events.
3) Predictive Modeling and Decision Support: The AI Algorithm Layer leverages machine learning models to predict the likelihood of specific side effects based on real-time data trends. For instance, rising IL-6 levels can predict the onset of CRS. The system's algorithms also assess the severity of predicted side effects, classifying them as mild, moderate, or severe. In response to these predictions, the system's decision support tools either suggest or automatically initiate emergency protocols, such as medication administration, tailored to the patient's history and current status.
4) Real-Time Alerts and Automated Actions: To ensure prompt intervention, the system includes a Monitoring and Alerting Layer that generates immediate alerts when data indicates an imminent side effect. These alerts are multi-modal, combining data from different sources to confirm the presence of a side effect before triggering a response. The system's dashboard interface provides an emergency protocol dashboard displaying suggested interventions and allows clinicians to approve or override AI recommendations. If authorized, the AI can trigger the administration of emergency medications, such as Epinephrine for anaphylaxis or Tocilizumab for CRS.
5) Emergency Protocols Integration: The system is equipped to handle a range of emergency scenarios, each with tailored responses. For Cytokine Release Syndrome (CRS), the AI issues an immediate alert if IL-6 or TNF-α levels spike and suggests administering Tocilizumab. In the case of neurotoxicity, the system recommends corticosteroids like Dexamethasone and anti-seizure medications. For other conditions, such as anaphylaxis, sepsis, Tumor Lysis Syndrome (TLS), Acute Respiratory Distress Syndrome (ARDS), and cardiac arrhythmias, the system provides specific medication suggestions and initiates appropriate protocols to manage the situation effectively.
6) Feedback, Learning, and Compliance: Post-event analysis is integral to the system's continuous improvement. After an emergency response, clinicians input the outcome, which the AI uses to refine its protocols. The system's adaptive learning capabilities allow it to continuously optimize its ability to predict and manage side effects, improving with each new data set. Additionally, the system ensures that all emergency protocols and patient data are handled securely, with detailed audit trails maintained for regulatory compliance.

Diagram 4. Comprehensive Monitoring and Data Acquisition: The system's architecture is designed to provide continuous and real-time monitoring of vital signs, cytokine levels, immune cell activity, and patient symptoms to detect early signs of adverse reactions such as Cytokine Release Syndrome (CRS), sepsis, or other critical conditions. The data acquisition process is meticulously structured to ensure the accurate collection and integration of data from various sources, including wearable sensors, blood analyzers, and clinician inputs.
1) Vital Signs Monitoring: The system integrates various wearable sensors and bedside monitors to continuously track key vital signs, including heart rate (HR), blood pressure (BP), respiratory rate (RR), and oxygen saturation (SpO2). These parameters are recorded at frequencies tailored to the patient's risk level, with high-risk patients receiving more frequent monitoring. The data is validated in real-time against the patient's baseline and expected physiological ranges, ensuring that any abnormal readings are immediately flagged for further analysis.
2) Cytokine Levels Monitoring: Real-time tracking of critical cytokines, such as IL-6, TNF-α, and IFN-γ, is essential for detecting the onset of a cytokine storm. The system uses automated blood analyzers to measure cytokine levels at regular intervals, typically ranging from every 30 minutes to every hour. These analyzers are integrated with the central system to ensure that results are immediately available for further analysis. The data is validated through calibration and cross-referencing with previous readings to ensure consistency and accuracy.
3) Immune Cell Activity Monitoring: The activity of immune cells, including dendritic cells and macrophages, is closely monitored using flow cytometry and tissue sampling. This allows the system to detect any dysfunction or abnormal polarization that could lead to adverse events. The data is uploaded in real-time, with specific markers tracked to assess immune cell function and polarization. The system also cross-validates immune cell activity data with cytokine levels to provide a comprehensive view of the patient's immune response.
4) Patient Symptoms Input: Clinicians play a crucial role in monitoring patient symptoms, such as fever, confusion, or difficulty breathing, which may indicate an adverse reaction. The system provides a user-friendly interface for healthcare providers to input these symptoms in real-time. Standardized symptom checklists ensure consistent reporting, and the data is immediately uploaded to the central monitoring system. The system's AI algorithms then correlate the reported symptoms with other real-time data, such as cytokine levels and immune cell activity, to identify potential side effects and assess their severity.
5) Data Integration and Synchronization: The system ensures that all collected data, whether from vital signs, cytokine levels, immune cell activity, or patient symptoms, is synchronized in real-time. This data is routed to a central monitoring hub where it is preprocessed, validated, and forwarded to the AI algorithm layer for further analysis and action. The AI-driven insights and alerts are then fed back to healthcare providers, allowing for immediate adjustments to monitoring protocols or treatment plans as needed.
6) Robust Data Storage and Security: To maintain the integrity and security of the data, the system employs real-time storage solutions with automatic backup protocols. All data transmissions are encrypted, ensuring patient confidentiality and compliance with regulatory standards. The system also implements redundancy protocols to prevent data loss in case of network issues, guaranteeing that critical information is always available when needed.

Diagram 5-1., 5-2., & 5-3. Data Preprocessing Layer for Side Effect Detection: The Data Preprocessing Layer is a critical component in ensuring that the raw data collected from various monitoring devices is transformed into clean, accurate, and reliable data for analysis by the AI system. This layer is designed to enhance the quality of the data through multiple stages of processing, including noise reduction, normalization, and data categorization. Each stage is essential for preparing the data to be effectively utilized in detecting and predicting potential side effects during T cell therapy.
1) Signal Processing and Noise Reduction: The initial step in the data preprocessing layer is signal processing, which aims to remove noise and artifacts from the raw data. Digital filters, such as Butterworth and Kalman filters, are implemented to eliminate high-frequency noise from vital signs data, including ECG and SpO2 readings. Adaptive filtering techniques further refine the signals by adjusting in real-time based on the incoming data characteristics. This ensures that motion artifacts and other distortions are minimized without compromising the integrity of the underlying signals. Signal smoothing techniques, such as moving average and wavelet transforms, are applied to highlight longer-term trends while preserving critical features of the data.
2) Normalization and Baseline Establishment: After noise reduction, the data undergoes normalization to ensure consistency across different measurements. The system establishes patient-specific baselines at the start of monitoring, considering factors like age, gender, and medical history. As the patient's condition evolves, the baselines are dynamically adjusted based on long-term trends and recent data. This process involves scaling the data to a fixed range using techniques like Min-Max scaling and Z-score normalization. Anomaly detection algorithms then flag any significant deviations from the normalized baselines, which could indicate potential side effects requiring further investigation.
3) Data Categorization and Time-Based Binning: Once the data is normalized, it is categorized into relevant indicators of potential side effects. For example, elevated levels of cytokines like IL-6 and TNF-α are categorized as potential indicators of Cytokine Release Syndrome (CRS). Neurological data, such as sudden changes in mental status or seizure activity, are classified as neurotoxicity indicators. The system also correlates manually inputted symptoms with physiological data to strengthen side effect identification. To facilitate trend analysis, the data is binned into fixed intervals, such as every 10 minutes or 1 hour. Adaptive binning techniques adjust the interval length based on the rate of change in the data, ensuring that rapid changes are captured accurately during critical periods.

Integration with the AI-Driven System: The preprocessed data is then fed into the AI Algorithm Layer, where it is analyzed for side effect prediction and decision-making. Real-time processing ensures that all signal processing, normalization, and categorization steps keep pace with the continuous data flow from the acquisition layer. The preprocessed data pipeline is designed to seamlessly integrate with the overall system, providing the AI layer with standardized and organized data that maximizes its ability to detect and respond to side effects efficiently. A feedback loop continuously refines the preprocessing algorithms based on the AI layer's outputs and clinical outcomes, ensuring that the system adapts to the evolving needs of T cell therapy monitoring.

Diagram 6. Emergency Response Protocols and AI-Driven Guidance: The AI-driven system is meticulously designed to identify and manage emergencies that may arise during or after cell therapy infusions, particularly in cancer patients. The system integrates continuous monitoring with real-time data analytics to promptly detect critical conditions such as Cytokine Release Syndrome (CRS), neurotoxicity, anaphylaxis, and other life-threatening events. By leveraging advanced predictive models, the system not only identifies potential emergencies but also provides clinicians with immediate recommendations on administering the appropriate rescue medications.
1) Cytokine Release Syndrome (CRS) Management: CRS is a common and potentially severe side effect of cell therapy, characterized by symptoms such as fever, hypotension, tachycardia, hypoxia, and multi-organ dysfunction. The system monitors cytokine levels and vital signs in real-time to detect early signs of CRS. Upon identifying the syndrome, the AI system recommends the administration of Tocilizumab, an IL-6 receptor antagonist, as the first-line treatment to block the inflammatory response. If symptoms persist or are severe, the system suggests corticosteroids such as Dexamethasone or Methylprednisolone to suppress the immune response further. Antipyretics and vasopressors are also recommended to manage fever and hypotension, respectively.
2) Neurotoxicity and Immune Effector Cell-Associated Neurotoxicity Syndrome (ICANS) Response: Neurotoxicity, particularly ICANS, can manifest as confusion, aphasia, seizures, headache, encephalopathy, and cerebral edema. The system continuously monitors neurological status and correlates it with immune activity to detect early signs of neurotoxicity. In cases of confirmed ICANS, the AI system advises the use of high-dose corticosteroids like Dexamethasone to reduce cerebral edema. Anti-seizure medications such as Levetiracetam are recommended to manage or prevent seizures, and in some cases, Mannitol is suggested to reduce intracranial pressure by osmotically drawing fluid from brain tissue.
3) Anaphylaxis and Acute Respiratory Distress Syndrome (ARDS) Management: For rapid-onset emergencies like anaphylaxis, characterized by difficulty breathing, hypotension, and swelling, the system prioritizes the administration of Epinephrine to constrict blood vessels and open airways. Antihistamines and corticosteroids are also recommended to counteract histamine effects and prevent biphasic anaphylaxis. In cases of ARDS, where patients experience severe shortness of breath and hypoxia, the system ensures immediate oxygen therapy and suggests corticosteroids to reduce lung inflammation. Sedatives may be recommended to ensure patient comfort and synchronization with mechanical ventilation.
4) Sepsis, Tumor Lysis Syndrome (TLS), and Cardiac Arrhythmias: Sepsis, a severe systemic infection, is another critical condition managed by the system. The AI algorithms detect early signs of sepsis through continuous monitoring of vital signs and suggest the immediate administration of broad-spectrum antibiotics like Piperacillin-Tazobactam. Vasopressors and intravenous fluids are also recommended to stabilize blood pressure and support organ function. For Tumor Lysis Syndrome, characterized by metabolic imbalances, the system advises the use of Allopurinol or Rasburicase to lower uric acid levels, along with loop diuretics and sodium bicarbonate to manage electrolyte disturbances and metabolic acidosis. In cases of cardiac arrhythmias, the system recommends medications such as Amiodarone, Adenosine, or Atropine, depending on the type of arrhythmia detected.
5) Correlations Between Symptoms and Rescue Medications: The system's decision-making process is underpinned by a sophisticated correlation engine that links specific symptoms with appropriate rescue medications. Immediate actions are prioritized for life-threatening conditions, ensuring that medications like Epinephrine, Amiodarone, and Tocilizumab are administered swiftly to stabilize the patient. The system also emphasizes the role of corticosteroids across multiple conditions to manage inflammation and immune responses. Supportive care medications, such as vasopressors, antibiotics, and oxygen, are recommended to maintain vital functions during systemic emergencies like sepsis and ARDS.

Diagram Representation and AI Algorithm Integration: The detailed diagram illustrates the integration of these emergency protocols within the AI-driven system. The predictive models and real-time data analytics are visually represented, showing the flow from data acquisition to emergency response recommendations. The diagram also highlights the correlation between specific symptoms and the corresponding rescue medications, ensuring that the AI system's suggestions are both timely and appropriate. This visual representation complements the descriptive explanation, providing a clear overview of how the system functions to enhance patient safety during cell therapy

Diagram 7. Feedback and Learning Layer: The Feedback and Learning Layer is integral to the adaptive nature of the AI-driven system, designed to continuously improve the accuracy and effectiveness of predicting, assessing, and managing side effects during cell therapy treatments. This layer focuses on integrating clinical feedback and post-event analysis to refine the AI algorithms, ensuring that the system evolves and adapts based on real-world data and outcomes.
1) Clinical Feedback Integration: A key component of this layer is the collection and integration of feedback from clinicians. The system automatically logs all adverse events, capturing detailed records of the circumstances leading up to the event, the interventions applied, and the patient outcomes. Clinicians are provided with an intuitive interface to input qualitative feedback on each event, including their observations, perceived causes of side effects, and the effectiveness of the interventions. This qualitative data is crucial for understanding the nuances of each case and for enhancing the system's ability to identify patterns that may not be evident from quantitative data alone.
2) Post-Event Analysis and Pattern Recognition: After an adverse event occurs, the system performs a retrospective analysis using machine learning algorithms to identify common patterns or predictors that may have been overlooked during real-time monitoring. Causal inference models are implemented to explore potential relationships between variables, such as a sudden increase in IL-6 leading to a cytokine storm. The system also assigns a severity score to each event based on clinical outcomes and the level of intervention required. Severe or unexpected events undergo root cause analysis (RCA) to identify system or process failures, ensuring that these issues are addressed and prevented in future cases.
3) Adaptive Learning and Continuous Model Updates: The system incorporates adaptive learning algorithms that enable it to update its predictive models incrementally as new data becomes available. This continuous learning process allows the system to evolve without needing to be retrained from scratch, integrating real-time feedback from clinicians directly into the model update process. The system also monitors for prediction errors, such as false positives or negatives, and adjusts the confidence levels of its predictions accordingly. This ensures that the AI becomes more cautious or assertive based on its historical performance and recent feedback.
4) Algorithm Refinement and Ensemble Learning: To further enhance predictive accuracy, the system employs hyperparameter tuning and model ensemble learning. By adjusting model hyperparameters, such as learning rates and regularization strength, the system optimizes its performance for the current patient population. Ensemble learning, which combines the outputs of multiple models (e.g., decision trees and neural networks), allows the system to select the best prediction strategy for each situation, improving overall decision-making and patient outcomes.
5) Outcome Tracking and Effectiveness Analysis: The system systematically tracks the effectiveness of AI-driven interventions, logging details such as medication dosage, timing, and patient responses. This data is used to conduct comparative analyses across different interventions and patient cohorts, identifying the most effective strategies for specific side effects or patient profiles. Key metrics, including patient survival rates, length of hospital stay, and morbidity rates, are tracked to assess the overall impact of AI-driven interventions on patient outcomes. This continuous tracking ensures that the system's models are refined based on real-world outcomes, leading to more effective and personalized patient care.

Integration with the Overall System: The Feedback and Learning Layer is seamlessly integrated with the AI Algorithm Layer, allowing for real-time adjustments to predictions and interventions as new data and feedback are received. The system facilitates collaborative decision-making between clinicians and the AI, ensuring that all feedback is transparent and easily accessible. This collaborative approach enables clinicians to make informed decisions based on the latest insights, with the AI system automatically incorporating feedback and outcome data into its algorithms for continuous improvement.

Diagram 8. Regulatory and Compliance Layer: The Regulatory and Compliance Layer is a critical component of the AI-driven system, designed to ensure that all patient data, particularly sensitive information related to immune monitoring and AI-driven decisions, is managed in full compliance with privacy laws and regulations such as HIPAA. This layer encompasses several key functions, including data privacy, encryption, audit trails, and transparency, each of which plays a vital role in maintaining the integrity and security of the system.
1) Data Privacy and HIPAA Compliance: The system employs advanced algorithms to classify and protect sensitive patient data, ensuring compliance with HIPAA and other relevant privacy laws. Data classification algorithms automatically identify and tag sensitive information, such as patient identifiers and immune function data, ensuring that this data is securely handled. The system implements role-based access control (RBAC), allowing only authorized personnel to access sensitive data, and multi-factor authentication (MFA) adds an extra layer of security. Additionally, the system prioritizes data minimization by anonymizing or de-identifying data where possible, particularly for datasets used in AI model training or analysis.
2) Encryption and Secure Data Handling: To safeguard data at all stages, the system uses industry-standard encryption protocols, such as AES-256, for both data at rest and in transit. End-to-end encryption ensures that data is encrypted at the source and only decrypted at the destination, preventing unauthorized access during transmission. The system also employs secure key management practices, including the use of hardware security modules (HSMs) for key storage and automatic key rotation to reduce the risk of key compromise. Data integrity is maintained through the use of cryptographic hashing algorithms and digital signatures, ensuring that data has not been tampered with during transmission or storage.
3) Comprehensive Audit Trails and Logging: The system maintains detailed, immutable logs of all AI-driven decisions, data access, and system activities to ensure transparency, accountability, and compliance with regulatory requirements. Comprehensive logging captures all significant events, including data access, AI-driven decisions, user actions, and system alerts, with each log entry including a timestamp, user ID, and detailed event description. Tamper-evident logging mechanisms, potentially using blockchain technology, prevent or detect unauthorized changes to the logs. Centralized log management allows for easy access, search, and analysis of log data, with automated alerts for any anomalies detected in the logs.
4) Transparency and Explainable AI (XAI): Transparency is a cornerstone of the system, achieved through the integration of Explainable AI (XAI) techniques that provide clear, human-readable explanations for all AI-driven decisions. This includes detailing the data inputs, the decision-making process, and the reasoning behind the AI's recommendations or actions. The system also allows users, such as clinicians, to provide feedback on the AI's explanations, which is used to refine the transparency mechanisms and improve future explanations. Users have easy access to decision logs, and regular audit reports summarize key activities, ensuring that all AI-driven processes are transparent and well-documented.
5) Integration with the Overall System: The Regulatory and Compliance Layer is integrated with real-time monitoring systems to ensure continuous oversight of all activities, including data handling, AI decisions, and system access. Feedback from the audit and logging systems is linked with the Feedback and Learning Layer, ensuring that any issues detected are addressed, and the AI system continues to improve in compliance with regulatory standards. A centralized dashboard provides compliance officers and administrators with a comprehensive view of the system's compliance status, allowing them to monitor, review, and generate reports as needed.

Diagram 9. Integration into Workflow Layer: The Integration into Workflow Layer is pivotal in ensuring that the AI-driven system seamlessly integrates into existing clinical practices, providing continuous monitoring, real-time risk assessments, and actionable therapeutic recommendations. This layer is designed to enhance patient safety, improve clinical outcomes, and foster a collaborative environment where AI and clinicians work together to deliver optimal care.
1) Continuous Patient Monitoring: The system's primary objective is to ensure continuous, real-time monitoring of critical immune parameters such as cytokine levels, dendritic cells, and macrophages. The integration of multi-source data aggregation allows for the collection and synchronization of data from various sources, including blood tests, flow cytometry, and macrophage activity assays. This real-time data pipeline is robust, ensuring that the data flows uninterruptedly from monitoring devices to the AI system. Dynamic data filtering and noise reduction algorithms are applied in real-time to ensure that only high-quality data enters the AI analysis pipeline, which is crucial for accurate trend detection and threshold monitoring. This seamless monitoring process ensures that the AI system has up-to-date information on the patient's immune status, enabling timely and accurate risk assessments and therapeutic recommendations.
2) AI Analysis and Risk Assessment: The system deploys AI models that continuously analyze the real-time data stream to predict the likelihood of immune-related complications such as cytokine storms or immune suppression. Predictive modeling takes into account current data, historical trends, and patient-specific factors to calculate a dynamic risk score that updates in real-time as new data is processed. Anomaly detection algorithms are employed to identify unusual patterns or deviations from expected trends, such as a sudden drop in dendritic cell activity. The AI system incorporates adaptive learning mechanisms, allowing it to refine its anomaly detection capabilities over time based on feedback from clinicians and outcomes of previous cases. The therapeutic recommendation engine generates personalized therapeutic recommendations based on the AI's analysis, providing clinicians with data-driven insights into the likely effects of various treatment options.
3) Therapeutic Decision-Making Support: The system integrates AI-driven analysis and recommendations into the clinical decision-making process, ensuring that clinicians have the information they need to make informed, timely decisions. Alerts are generated based on the severity of the predicted side effect or immune complication, and are communicated through multiple channels to ensure that clinicians are promptly informed. The decision support interface presents AI-driven suggestions alongside the current patient data and risk assessments, designed to support quick, informed decision-making. Explainability features are included to allow clinicians to understand the reasoning behind the AI's recommendations, ensuring transparency and trust in the AI system. The system also allows for customizable alerts and suggestions, enabling clinicians to tailor the system to their specific needs and preferences.
4) Decision Logging and Outcome Feedback: The system automatically logs all clinical decisions made based on AI-driven suggestions, including the data and risk assessments that informed those decisions. This comprehensive audit trail records every interaction with the AI system, providing an immutable record that is easily accessible for review. As treatments are administered, the system tracks and documents immediate outcomes, feeding this data back into the AI system to refine future predictions and recommendations. Long-term monitoring of patient outcomes is also implemented, allowing the AI system to learn from the full range of patient responses and adjust its models accordingly. A continuous feedback loop is established, where the outcomes of AI-driven decisions are used to refine and improve the AI's predictive models and decision support algorithms, leading to more accurate predictions and better patient care over time.

System Integration and Scalability: To ensure seamless integration into clinical workflows, the system is designed with real-time synchronization, enabling all layers of the AI-driven system to operate in harmony. User-centric design principles guide the development of interfaces and workflows, reducing the learning curve for clinicians and enhancing adoption. The system's architecture is scalable and flexible, allowing for future enhancements such as the integration of additional data sources, new predictive models, and expanded decision support capabilities. This ensures that the system remains relevant and effective as medical practices and technologies evolve.

### DETAILED DESCRIPTION OF THE INVENTION

### 1. System Architecture Overview

### 1.1. Objective

To provide a detailed and comprehensive overview of the system architecture, emphasizing the unique integration of AI-driven data analytics, multi-modal data inputs, and adaptive learning mechanisms designed to optimize engineered T cell therapies in patients with cancer, autoimmune diseases, and inflammatory conditions.

### 1.2. Detailed Explanation

The AI-driven data analytics system for optimizing engineered T cell therapies is designed as a multi-layered architecture that seamlessly integrates with existing clinical workflows. Each layer within this architecture has a distinct and critical role in ensuring the system's overall functionality. The interaction between these layers facilitates seamless data flow, real-time analysis, and informed decision-making, ultimately enhancing the safety and effectiveness of T cell therapies. The architecture is structured to allow for continuous monitoring of patient data, predictive analytics, and dynamic therapeutic adjustments. By integrating data from diverse sources—such as patient monitoring systems, laboratory tests, and imaging studies—the system creates a comprehensive, real-time picture of the patient's condition. This multi-layered approach ensures that data is processed efficiently and accurately, enabling the AI algorithms to make timely and reliable predictions about potential complications, such as Cytokine Release Syndrome (CRS) or Tumor Lysis Syndrome (TLS). The system's design not only supports proactive management of these risks but also integrates feedback mechanisms that allow it to evolve and improve over time, maintaining its relevance in the rapidly advancing field of T cell therapy. So, This integrated approach not only enhances the system's predictive accuracy but also ensures that therapeutic recommendations are personalized and adaptive to each patient's unique physiological responses.

### 2. Data Acquisition Layer

The Data Acquisition Layer is the foundational component of the AI-driven data analytics system, responsible for gathering real-time data from multiple sources, including patient monitoring systems, laboratory tests, imaging studies, and wearable devices. This layer is crucial for ensuring comprehensive monitoring of patient physiology during and after T cell therapies, providing the essential data that underpins all subsequent analysis and decision-making within the system.

### 2.1. Objective

To outline the robust and comprehensive data acquisition capabilities of the system, highlighting its ability to continuously collect and integrate high-frequency, multi-modal data from diverse sources, thereby ensuring comprehensive, real-time monitoring of patient physiology throughout the T cell therapy process.

### 2.2. Function

The Data Acquisition Layer is responsible for the continuous collection and integration of real-time data from a wide array of sources, including but not limited to patient monitoring systems, laboratory information systems (LIS), imaging modalities (such as MRI, CT, and PET scans), wearable devices, and genomic profiling tools. This layer forms the backbone of the system, ensuring that all relevant physiological and pathological data is captured in real-time for immediate processing and analysis.

### 2.3. Components

The Data Acquisition Layer is equipped with advanced interfaces for seamless integration with a variety of clinical data sources. These include:

### 2.3.1Vital Signs Monitors

Continuously monitor and transmit data on heart rate, blood pressure, respiratory rate, oxygen saturation, and electrocardiogram (ECG) readings.

2.3.2Laboratory Systems

Real-time integration with laboratory systems for monitoring critical biomarkers such as cytokine levels (e.g., IL-6, TNF-α, IFN-γ), electrolyte balances, and immune cell counts (e.g., CD4+, CD8+T cells).

### 2.3.3Imaging Systems

Integration with radiological imaging modalities (MRI, CT, PET) for ongoing assessment of tumor status, tissue integrity, and treatment response.

### 2.3.4Wearable Devices

Continuous monitoring of patient activity levels, glucose levels, sleep patterns, and other metabolic parameters through wearable devices.

### 2.3.5Genomic and Proteomic Tools

Integration with advanced genomic and proteomic profiling tools to capture patient-specific molecular data critical for personalized treatment strategies.

### 2.4. Detailed Explanation

The Data Acquisition Layer is a pivotal component of the AI-driven data analytics system, tasked with gathering and integrating a comprehensive range of real-time patient data. This layer ensures that the system receives continuous, high-fidelity data inputs from multiple, diverse sources, which are crucial for the accurate monitoring, analysis, and optimization of engineered T cell therapies. By interfacing seamlessly with various clinical data sources—including patient monitoring systems, laboratory tests, imaging studies, wearable devices, and genomic profiling tools—this layer ensures that all relevant physiological and pathological data is captured in real-time, thereby enabling immediate and effective data processing and analysis. The integration of such diverse data streams into a unified platform allows the system to provide a comprehensive, up-to-the-minute view of the patient's condition, facilitating precise, timely, and personalized therapeutic interventions.

### 2.5. Patient Monitoring Systems

### 2.5.1. Real-Time Vital Signs Monitoring

This component continuously collects vital signs such as heart rate (HR), blood pressure (BP), respiratory rate (RR), and oxygen saturation (SpO2). These parameters are essential for assessing the patient's overall condition and detecting early signs of adverse reactions, such as CRS.

### 2.5.2. ECG Monitoring

Electrocardiogram (ECG) data is captured to monitor the heart's electrical activity, which is vital for detecting arrhythmias or other cardiac events that may occur during therapy.

### 2.5.3. Centralized Monitoring

All vital signs data are transmitted to a centralized monitoring system where they are continuously analyzed for trends that could indicate the onset of adverse events. The system allows for the integration of data from multiple monitoring devices, ensuring a comprehensive overview of the patient's physiological status.

### 2.5.4. Laboratory Data Integration

### 2.5.4.1. Cytokine Level Monitoring

The system integrates data from laboratory assays measuring cytokine levels, such as IL-6, IL-10, TNF- [symbol] ±, and IFN- [symbol] ≥. These biomarkers are crucial for predicting the onset of Cytokine Release Syndrome (CRS) and other inflammatory responses, enabling early detection and intervention.

### 2.5.4.2. Blood Count and Metabolic Panel Integration

Complete Blood Count (CBC) data, including immune cell counts (e.g., CD4+, CD8+ T cells), and Comprehensive Metabolic Panel (CMP) results are integrated into the system. This integration is vital for monitoring the patient's immune function and metabolic status, providing a comprehensive view of the patient's health and aiding in the early detection of adverse events.

### 2.5.4.3. Automated Data Input

Lab results are automatically fed into the system in real-time, minimizing delays and ensuring that the most current information is always available for analysis. The system supports bidirectional communication with laboratory systems, allowing for both data retrieval and feedback on analysis.

### 2.5.5. Imaging Systems

### 2.5.5.1. Radiological Data Integration

Imaging studies, including MRI, CT scans, PET scans, and Voxel-Based Imaging are integrated into the system to provide insights into the anatomical and functional status of the tumor and surrounding tissues. This data is essential for assessing the effectiveness of T cell therapy and making necessary adjustments to treatment protocols.

### 2.5.5.2. Automated Imaging Analysis

The system includes capabilities for the automated analysis of imaging data, such as measuring tumor size and monitoring treatment response. This allows for real-time adjustments to therapy based on the latest imaging results, improving the precision of treatment interventions.

### 2.5.6. Wearable Devices

### 2.5.6.1. Continuous Glucose Monitoring

For patients at risk of metabolic disturbances, continuous glucose monitors (CGMs) provide real-time data on blood glucose levels. This data is integrated into the system to help manage risks such as Tumor Lysis Syndrome (TLS) and other metabolic complications.

### 2.5.6.2. Activity and Sleep Tracking

Wearable devices that track physical activity, sleep patterns, and other lifestyle factors are also integrated into the system. This data helps clinicians understand how the patient's overall lifestyle and daily habits impact their response to T cell therapy.

### 2.5.6.3. Data Transmission and Synchronization

Data from wearable devices is transmitted wirelessly and synchronized with other patient data in the system, ensuring that all relevant information is available for real-time analysis. The system supports the integration of multiple wearable devices, providing a holistic view of the patient's health and activity levels.

### 2.6. Data Integration and Standardization

The Data Acquisition Layer is designed to handle large volumes of data from diverse sources, ensuring that each data point is accurately recorded, standardized, and made available for real-time analysis. The system supports a variety of data formats and integrates with existing clinical systems, minimizing the need for manual data entry and reducing the risk of errors. This standardization process is crucial for ensuring that the AI-driven analysis is based on accurate and consistent data, which in turn improves the reliability of the system's predictions and recommendations.

### 3. Data Preprocessing Layer

The Data Preprocessing Layer plays a crucial role in transforming raw data into a format that is ready for accurate and reliable analysis by the AI algorithms. This layer is responsible for cleaning, standardizing, and categorizing data to ensure consistency and accuracy, which are essential for precise AI-driven predictions and recommendations. By addressing issues such as noise, variability, and missing data, the Data Preprocessing Layer lays the foundation for high-quality analysis, ultimately improving the outcomes of T cell therapies.

### 3.1. Objective

To establish a rigorous framework for ensuring that all data entering the AI-driven analytics system is of the highest quality, this layer is tasked with performing advanced preprocessing functions, including data cleaning, standardization, categorization, and imputation, thereby enabling precise and reliable AI analysis.

### 3.2. Function

The Data Preprocessing Layer is responsible for transforming raw, heterogeneous data into a standardized format that is ready for AI-driven analysis. This includes advanced signal processing, noise reduction, normalization, and categorization to ensure data consistency, accuracy, and relevance across all sources.

### 3.3. Components

The Data Preprocessing Layer employs state-of-the-art techniques and algorithms, including:

### 3.3.1. Signal Processing and Noise Reduction

Application of advanced filtering techniques (e.g., Butterworth, Kalman filters) to eliminate noise and artifacts from vital signs, ECG, and other high-frequency data streams, ensuring that only clean, high-fidelity data is processed.

### 3.3.2. Normalization and Standardization

Implementation of z-score normalization, min-max scaling, and other techniques to standardize data across different measurement scales and patient baselines, ensuring comparability and consistency.

### 3.3.3. Data Imputation and Smoothing

Utilization of machine learning algorithms and statistical methods (e.g., linear interpolation, predictive modeling) to address missing or inconsistent data points, maintaining the integrity of the data set.

### 3.3.4. Data Categorization

Categorization of data into relevant biomarkers, time-based bins, and event-based classifications, facilitating targeted analysis and trend detection.

### 3.3.5. Signal Processing

### 3.3.5.1. Noise Reduction

The system employs advanced filtering techniques to remove noise and artifacts from the raw signals collected by patient monitoring devices and wearable sensors. For example, Butterworth or Kalman filters are used to smooth ECG signals, eliminating high-frequency noise that could interfere with accurate heart rate monitoring.

### 3.3.5.2. Signal Smoothing

To further enhance data quality, the system applies signal smoothing techniques such as moving averages or wavelet transforms. These methods reduce the impact of short-term fluctuations and highlight longer-term trends that are more relevant for clinical decision-making. For instance, smoothing glucose monitoring data helps identify consistent patterns rather than reacting to transient spikes.

### 3.3.6. Normalization

### 3.3.6.1. Baseline Establishment

The system establishes a baseline for each patient by analyzing initial data collection. This baseline reflects the patient's normal physiological ranges and serves as a reference point for detecting significant deviations. For example, baseline cytokine levels (e.g., IL-6) are established during the initial monitoring phase, allowing the system to detect abnormal elevations during therapy. 3.3.6.2. Standardization: Data from different sources and devices are standardized using techniques such as z-score normalization or min-max scaling. This process ensures that variations in measurement scales (e.g., blood pressure vs. glucose levels) do not affect the analysis. Standardization allows the AI algorithms to consistently compare and analyze data across different patients and treatment sessions.

### 3.3.7. Data Imputation

### 3.3.7.1. Handling Missing Data

In cases where data points are missing due to device malfunctions or communication errors, the system uses data imputation techniques to estimate and fill in the gaps. For example, linear interpolation or predictive modeling may be applied to estimate missing cytokine levels based on trends in available data.

### 3.3.7.2. Consistency Checks

The system performs consistency checks to identify and correct anomalies or outliers in the data. For instance, if a sudden drop-in heart rate is detected but not corroborated by other vital signs, the system may flag this as a potential error and either request verification or apply imputation techniques to correct the data.

### 3.3.8. Data Categorization

### 3.3.8.1. Biomarker Classification

The system categorizes data into specific biomarkers, such as cytokine levels (e.g., IL-6, TNF-α), immune cell counts (e.g., CD4+, CD8+), and vital signs (e.g., HR, BP). This classification is essential for targeted analysis, allowing the AI algorithms to focus on relevant indicators of adverse events. Each biomarker is tagged with metadata, including its source, collection time, and measurement units, to facilitate accurate analysis.

### 3.3.8.2. Time-Based Binning

Data is organized into time bins (e.g., every 10 minutes, 30 minutes, 1 hour) to track changes over time. This temporal organization allows the system to analyze trends and patterns, such as the gradual increase in cytokine levels that may signal the onset of CRS. The time-based binning is adjustable based on the clinician's needs and the specific requirements of the treatment protocol.

### 3.3.9. Event-Based Data Classification

### 3.3.9.1. Trigger Events

The system classifies data based on specific clinical events, such as the administration of a therapeutic dose, the onset of a symptom, or a scheduled lab test. This event-based classification helps in understanding the relationship between interventions and patient outcomes. For example, the system can correlate the timing of a T cell infusion with subsequent changes in cytokine levels to assess the therapy's impact.

### 3.3.9.2. Event Markers

Each data point associated with a clinical event is tagged with an event marker, indicating its relevance to specific therapeutic actions or patient responses. These markers are used by the AI algorithms to identify cause-and-effect relationships and adjust treatment recommendations accordingly.

### 3.4. Detailed Explanation

The Data Preprocessing Layer is a critical component of the AI-driven system, designed to ensure that all data is thoroughly cleaned, standardized, and prepared before entering the AI algorithms. This layer addresses common challenges such as noise, variability, and missing data by applying advanced signal processing techniques, normalization algorithms, and imputation methods. By standardizing data from diverse sources—such as patient monitoring systems, laboratory tests, and wearable devices—this layer ensures that the AI algorithms receive high-quality, consistent data inputs, which are essential for generating accurate and reliable predictions. The data categorization process further enhances the system's ability to detect trends and patterns, enabling more precise and targeted analysis that directly informs therapeutic decision-making.

### 4. AI Algorithm Layer

The AI Algorithm Layer is the core of the system, where advanced machine learning models and decision support systems analyze preprocessed data to predict adverse events, assess their severity, and provide therapeutic recommendations. This layer transforms raw data into actionable insights through sophisticated predictive modeling and decision support tools, enabling clinicians to make informed decisions about patient care.

### 4.1. Objective

The objective of the AI Algorithm Layer is to describe the advanced machine learning models and decision support systems that analyze preprocessed data to predict adverse events, assess their severity, and provide therapeutic recommendations. This layer is critical for enabling real-time, data-driven insights and optimizing treatment protocols and recommendation to clinicians during and after T cell therapies.

### 4.2. Function

The AI Algorithm Layer is responsible for processing the preprocessed data through a suite of sophisticated machine learning and deep learning algorithms, designed to predict the likelihood of adverse events such as Cytokine Release Syndrome (CRS), Tumor Lysis Syndrome (TLS), and other immune-related complications. The algorithms continuously analyze multi-modal data inputs to provide early detection and personalized therapeutic recommendations.

### 4.3. Components

This layer integrates a diverse array of AI models and techniques, including:

### 4.3.1. Long Short-Term Memory (LSTM) Networks

Used for analyzing time-series data to detect trends and predict future adverse events based on historical patient data.

### 4.3.2. Random Forests

Employed for classification tasks, particularly in identifying patients at high risk for specific complications based on a combination of biomarkers and clinical variables.

### 4.3.3. Convolutional Neural Networks (CNNs)

Applied to imaging data and multi-dimensional datasets, enabling the system to identify complex patterns and correlations that may not be apparent through traditional analysis methods.

### 4.3.4. Reinforcement Learning

Incorporates adaptive learning mechanisms that refine the predictive models over time, continuously improving the system's accuracy and relevance based on real-world outcomes.

### 4.3.5. Fuzzy Logic Algorithms

Utilized for decision-making processes where uncertainty and imprecision are inherent, allowing the system to generate nuanced and context-aware recommendations.

### 4.4. Detailed Explanation

The AI Algorithm Layer is the core analytical engine of the system, driving its ability to predict adverse events and support clinical decision-making. By applying a combination of machine learning, deep learning, and reinforcement learning techniques, the system can analyze complex, multi-dimensional data to generate real-time insights that are tailored to each patient's unique physiological profile. The integration of LSTM networks, Random Forests, CNNs, and other AI models allows the system to continuously adapt to new data, refine its predictions, and improve its therapeutic recommendations. The use of fuzzy logic further enhances the system's ability to make informed decisions in situations where data is uncertain or incomplete, ensuring that clinicians receive the most relevant and actionable guidance possible.

### 4.4.1. Predictive Models

### 4.4.1.1. Machine Learning Algorithms

The system utilizes supervised learning models such as Random Forests, Gradient Boosting Machines (GBMs), and Support Vector Machines (SVMs), which are trained on historical patient data to predict the likelihood of adverse events like CRS and TLS. These models analyze input features, such as cytokine levels and vital signs, to generate probability scores for potential adverse events.

### 4.4.1.2. Deep Learning Models

For more complex pattern recognition, the system employs deep learning models, including Convolutional Neural Networks (CNNs) and Long Short-Term Memory (LSTM) networks. These models are particularly effective in analyzing multi-dimensional data, such as time-series trends in cytokine levels and ECG signals, to detect subtle changes that may precede clinical symptoms.

### 4.4.2. Time-series Analysis

### 4.4.2.1. ARIMA Models

AutoRegressive Integrated Moving Average (ARIMA) models are used to forecast future values of critical biomarkers based on historical data. This time-series analysis is crucial for predicting the onset of adverse events by identifying trends and patterns in the data over time.

### 4.4.2.2. LSTM Networks

Long Short-Term Memory networks are deployed to capture temporal dependencies in the data, allowing the system to understand how current conditions are influenced by previous states. This is particularly useful in predicting delayed adverse reactions.

### 4.4.3. Severity Assessment

### 4.4.3.1. Multi-Class Classification

The system assigns severity scores to predicted adverse events using multi-class classification models. For example, the system may classify CRS as mild, moderate, or severe based on the magnitude of cytokine elevation, vital sign abnormalities, and patient-specific factors.

### 4.4.3.2. Threshold-Based Classification

The system uses predefined thresholds for key biomarkers, such as IL-6 levels or blood pressure, to classify the severity of an event. These thresholds can be dynamically adjusted based on patient history and ongoing clinical data.

### 4.4.4. Risk Stratification

### 4.4.4.1. Risk Scores

The system generates a risk score for each patient, reflecting the likelihood and potential severity of adverse events. This score is continuously updated as new data is processed, allowing clinicians to prioritize interventions based on real-time risk assessments.

### 4.4.4.2. Scenario Simulation

The system simulates different clinical scenarios to assess how the severity of an adverse event may evolve. For example, it may predict how cytokine levels will respond to a specific intervention, helping clinicians make informed decisions.

### 4.4.5. Decision Support

### 4.4.5.1. Therapeutic Recommendations

Based on the analysis of real-time data, the system provides personalized treatment recommendations, such as adjusting the dosage of immunosuppressive drugs or administering specific cytokine inhibitors. These recommendations are tailored to the individual patient's condition and history.

### 4.4.5.2. Drug Interaction Alerts

The system also analyzes potential drug interactions, alerting clinicians to any risks associated with the recommended therapies. This ensures that the treatment plan is both effective and safe.

### 4.4.6. Real-Time Decision Making

### 4.4.6.1. Interactive Decision Support

The system includes an interactive decision support tool that allows clinicians to explore different treatment options and their predicted outcomes. Clinicians can adjust parameters such as drug dosage or timing, and the system will provide updated predictions based on these changes.

### 4.4.6.2. Adaptive Protocols

The system can suggest adaptive treatment protocols that evolve based on the patient's response to therapy. For example, if a patient's condition improves, the system may recommend tapering certain medications while escalating therapy if the condition worsens.

### 4.4.7. Continuous Learning and Adaptation

### 4.4.7.1. Feedback Integration

The AI algorithms continuously integrate feedback from clinical outcomes and clinician input, refining their predictive models and decision support capabilities. This feedback loop ensures that the system adapts to new data and evolving clinical practices, maintaining its relevance and accuracy over time.

### 4.4.7.2. Adaptive Learning

The system employs adaptive learning techniques, such as reinforcement learning, to improve its performance based on real-world experience. For example, the system may adjust its weighting of certain biomarkers if clinical outcomes indicate that they are more or less predictive of adverse events than initially modeled.

### 5. Monitoring and Alerting Layer

The Monitoring and Alerting Layer is a critical component of the AI-driven system, designed to provide real-time alerts and visualization tools that enable clinicians to continuously monitor patient status. This layer also offers interactive decision-making tools, allowing clinicians to respond promptly and effectively to alerts with appropriate interventions. By continuously informing clinicians of the patient's status and alerting them to any significant changes, this layer plays a vital role in maintaining patient safety and optimizing therapeutic outcomes.

### 5.1. Objective

To provide a robust, real-time monitoring and alerting framework that empowers clinicians to manage patient care with precision, by leveraging continuous data monitoring, predictive analytics, and interactive decision-making tools to preemptively address potential complications in engineered T cell therapies.

### 5.2. Function

The Monitoring and Alerting Layer is designed to continuously track and analyze patient data in real-time, generating immediate alerts when predefined safety thresholds are exceeded or when the AI algorithms predict a high likelihood of adverse events such as Cytokine Release Syndrome (CRS), Tumor Lysis Syndrome (TLS), or neurotoxicity. This proactive monitoring system enables clinicians to intervene early, mitigating risks and optimizing therapeutic outcomes.

### 5.3. Components

The Monitoring and Alerting Layer includes several critical pillars:

### 5.3.1. Real-Time Alerts

The system generates two types of alerts:

### 5.3.1.1. Threshold-Based Alerts

Triggered when specific biomarkers or vital signs exceed established safety limits (e.g., elevated IL-6 levels or rapid changes in heart rate).

### 5.3.1.2. Predictive Alerts

Generated based on AI-driven predictions of imminent adverse events, allowing for early intervention before clinical symptoms manifest.

### 5.3.2. Dashboard Interface

A user-friendly interface that provides clinicians with real-time visualizations of patient data trends, risk assessments, and predictive analytics. The dashboard allows for interactive exploration of data, enabling clinicians to drill down into specific parameters and understand the underlying factors driving alerts.

5.3.3. Decision-Making Tools

Interactive tools that allow clinicians to explore “what-if” scenarios, simulate the effects of potential interventions, and receive AI-driven recommendations tailored to the patient's current status and historical data.

### 5.4. Detailed Explanation

The Monitoring and Alerting Layer is integral to the system's ability to maintain patient safety and optimize therapeutic outcomes in engineered T cell therapies. This layer continuously monitors patient data, utilizing advanced AI algorithms to detect trends, patterns, and potential risks. When critical thresholds are crossed or when the system predicts a high probability of an adverse event, real-time alerts are generated, providing clinicians with the information they need to take immediate action. The dashboard interface offers a comprehensive, real-time view of patient status, while the decision-making tools enable clinicians to interact with the data, explore potential interventions, and make informed decisions that are supported by robust predictive analytics. This layer ensures that clinicians are always aware of the patient's condition and can respond proactively to emerging risks, thereby enhancing the overall safety and efficacy of T cell therapies.

### 5.4.1. Real-Time Alerts

### 5.4.1.1. Threshold-Based Alerts

5.4.1.1.1. Immediate Notification: When any biomarker or vital sign crosses a predefined safety threshold (e.g., IL-6 levels exceeding 100 pg/mL), the system generates an immediate alert. These alerts are designed to be highly visible and can be delivered through multiple channels, including desktop notifications, SMS, and mobile app alerts.

### 5.4.1.1.2. Customizable Thresholds

Clinicians can customize the thresholds for different parameters based on the patient's condition and treatment goals. This allows for personalized monitoring that adapts to the specific needs of each patient.

### 5.4.2. Predictive Alerts

### 5.4.2.1. Early Warning System

The system's predictive models generate alerts not only when thresholds are crossed but also when the likelihood of an adverse event exceeds a certain probability. For example, if the system predicts a 70% chance of CRS based on rising IL-6 levels and declining blood pressure, it will issue a predictive alert even before symptoms become apparent.

### 5.4.2.2. Multi-Modal Alerts

The system combines data from different sources (e.g., cytokine levels, ECG data, and patient-reported symptoms) to generate comprehensive alerts. This multi-modal approach ensures that alerts are based on a holistic view of the patient's condition, reducing the likelihood of false positives.

### 5.4.3. Dashboard Interface

### 5.4.3.1. Real-Time Visualization

### 5.4.3.1.1. Data Trends and Patterns

The dashboard provides real-time graphs and charts that display trends in vital signs, cytokine levels, and other critical biomarkers. Clinicians can easily spot patterns that may indicate the onset of adverse events, such as a gradual increase in IL-6 levels over several hours.

### 5.4.3.1.2. Interactive Graphs

The system allows clinicians to interact with the data by zooming in on specific time periods, comparing different biomarkers, and overlaying patient events (e.g., medication administration) onto the graphs to see how treatments are affecting the patient's condition.

### 5.4.4. Customizable Layout

### 5.4.4.1. Personalized Dashboards

Clinicians can customize the dashboard layout to focus on the parameters most relevant to their patient's care. For example, an oncologist might prioritize cytokine trends and immune cell counts, while a cardiologist might focus on ECG data and blood pressure trends.

5.4.4.2. Integration with Electronic Health Records (EHR)

The dashboard integrates seamlessly with the hospital's EHR system, allowing clinicians to access the patient's full medical history alongside real-time data. This integration facilitates comprehensive care planning and documentation.

### 5.4.5. Interactive Tools

### 5.4.5.1. Treatment Simulation

### 5.4.5.1.1. Scenario Analysis

The system includes tools for simulating the impact of different treatment options. For instance, clinicians can input a hypothetical change in drug dosage, and the system will predict the likely effects on cytokine levels, immune response, and overall patient status.

### 5.4.5.1.2. What-If Scenarios

Clinicians can explore “what-if” scenarios to evaluate potential outcomes. For example, they might simulate the effects of delaying a medication dose by one hour or administering an additional immunosuppressant, with the system providing predictions based on these inputs.

### 5.4.5.2. Clinical Decision-Making Support

### 5.4.5.2.1. Real-Time Adjustments

The system allows clinicians to make real-time adjustments to treatment protocols directly from the dashboard. For example, if the system alerts to a rising risk of CRS, the clinician can immediately order a reduction in T cell infusion rates or initiate cytokine blockade therapy.

### 5.4.5.2.2. Collaboration Tools

The dashboard includes features for collaboration, such as shared notes and instant messaging, enabling the care team to communicate and coordinate responses to alerts and treatment decisions in real-time.

### 6. Feedback and Learning Layer

The Feedback and Learning Layer is designed to ensure that the AI-driven system continuously improves its performance by incorporating clinical feedback and patient outcomes into its models. This layer plays a crucial role in adapting the system to new data, refining its predictive accuracy, and enhancing its decision-making capabilities over time. By learning from each interaction, the system evolves to better serve the needs of clinicians and patients.

6.1.

### Objective

To establish dynamic feedback and learning mechanism that continuously enhances the system's predictive accuracy, decision-making capabilities, and therapeutic efficacy by incorporating real-world clinical feedback, patient outcomes, and adaptive learning into the AI-driven analytics.

### 6.2. Function

The Feedback and Learning Layer is designed to systematically improve the system's performance over time by integrating clinical feedback, analyzing patient outcomes, and updating the AI algorithms accordingly. This continuous learning process ensures that the system remains current, effective, and aligned with the latest clinical evidence and treatment practices.

### 6.3. Components

The Feedback and Learning Layer consists of several key components:

### 6.3.1. Post-Event Analysis

The system conducts detailed reviews of adverse immune responses, such as CRS and TLS, by analyzing the sequence of events leading up to the adverse reaction, including patient data, clinical interventions, and AI-generated recommendations. This analysis identifies areas for improvement and informs subsequent model adjustments.

### 6.3.2. Adaptive Learning Algorithms

Machine learning techniques, including reinforcement learning, are employed to update and refine the predictive models based on new data and outcomes. The system's algorithms continuously evolve, incorporating real-world evidence and clinician input to enhance predictive accuracy and therapeutic recommendations.

### 6.3.3. Outcome Tracking

The system tracks the effectiveness of interventions over time, monitoring long-term patient outcomes, including survival rates, quality of life, and recurrence of adverse events. This data is used to validate and refine the AI-driven recommendations, ensuring that they are evidence-based and clinically relevant.

### 6.4. Detailed Explanation

The Feedback and Learning Layer is essential for maintaining and improving the system's performance, ensuring that it evolves in response to real-world clinical experiences and outcomes. By conducting post-event analyses, the system identifies patterns and factors that contribute to adverse events, enabling targeted improvements to the predictive models. The adaptive learning algorithms allow the system to continuously refine its predictions and recommendations based on new data, clinical feedback, and evolving treatment paradigms. Outcome tracking further strengthens this layer by providing a comprehensive understanding of the long-term effects of the system's recommendations, ensuring that they remain aligned with best practices and the latest clinical evidence. This continuous learning process ensures that the system remains an effective and reliable tool for optimizing engineered T cell therapies.

### 6.4.1. Post-Event Analysis

### 6.4.1.1. Adverse Event Review

### 6.4.1.1.1. Comprehensive Data Review

After an adverse event, such as Cytokine Release Syndrome (CRS) or Tumor Lysis Syndrome (TLS), the system conducts a comprehensive review of all relevant data, including cytokine levels, vital signs, and treatment decisions leading up to the event. This review helps identify any early warning signs or missed opportunities for intervention.

### 6.4.1.1.2. Root Cause Analysis

The system uses machine learning techniques to perform root cause analysis, identifying the factors that most likely contributed to the adverse event. For example, it might pinpoint a specific cytokine threshold that, when crossed, consistently precedes CRS.

### 6.4.1.2. Clinical Feedback Integration

### 6.4.1.2.1. Clinician Input

The system allows clinicians to provide feedback on its performance, such as whether alerts were timely and accurate and if the recommended interventions were effective. This feedback is crucial for refining the system's algorithms and improving future performance.

### 6.4.1.2.2. Post-Event Reporting

The system generates detailed post-event reports that are shared with the clinical team. These reports include an analysis of the event, recommendations for future monitoring, and suggestions for adjusting the system's predictive models.

### 6.4.2. Adaptive Learning

### 6.4.2.1. Continuous Model Updates

### 6.4.2.1.1. Real-Time Learning

The system's machine learning models are continuously updated based on new data and outcomes. This real-time learning ensures that the system remains up to date with the latest clinical evidence and treatment practices.

### 6.4.2.1.2. Outcome-Based Refinement

The system adjusts its algorithms based on the success or failure of its predictions and recommendations. For example, if a specific predictive model consistently underestimates the risk of CRS, the system will adjust the model's parameters to improve its accuracy.

### 6.4.2.2. Patient-Specific Customization

### 6.4.2.2.1. Personalized Learning

The system learns from each patient's unique response to therapy, allowing it to tailor its predictions and recommendations to the individual. For example, if a patient's cytokine levels tend to rise rapidly after T cell infusion, the system will adjust its predictive models to account for this pattern.

### 6.4.2.2.2. Longitudinal Data Analysis

The system analyzes data over the long term, identifying trends that may only become apparent after several treatment cycles. This long-term learning is crucial for managing chronic conditions and optimizing ongoing therapy.

### 6.4.3. Outcome Tracking

### 6.4.3.1. Effectiveness Monitoring

### 6.4.3.1.1. Intervention Success Tracking

The system tracks the effectiveness of each intervention, such as the administration of cytokine inhibitors or adjustments to T cell infusion rates. This tracking helps determine which interventions are most successful in managing specific adverse events.

### 6.4.3.1.2. Long-Term Outcome Monitoring

The system also tracks long-term patient outcomes, such as overall survival and quality of life, to assess the impact of T cell therapy and the system's recommendations on patient health.

### 6.4.3.2. Feedback Loop

### 6.4.3.2.1. Continuous Improvement

The system uses outcome data to refine its predictive models and decision support tools continuously. This feedback loop ensures that the system becomes more accurate and effective over time.

### 6.4.3.2.2. Clinician-Driven Updates

Clinicians can directly influence the system's learning process by providing feedback on the success of different interventions and suggesting areas for improvement. This clinician-driven feedback is essential for keeping the system aligned with real-world clinical practices.

### 7. Regulatory and Compliance Layer

The Regulatory and Compliance Layer is essential for ensuring that the AI-driven system adheres to all relevant legal and regulatory standards, particularly those related to data privacy, security, and transparency. This layer is crucial for protecting patient data, ensuring transparency in decision-making, and maintaining compliance with regulations such as the Health Insurance Portability and Accountability Act (HIPAA). By implementing rigorous data handling protocols, encryption, audit trails, and transparency measures, this layer ensures that the system can be safely and legally deployed in clinical settings.

### 7.1. Objective

To establish a robust regulatory and compliance framework that ensures the system adheres to all relevant legal, ethical, and regulatory standards, particularly those related to data privacy, security, and transparency, such as HIPAA compliance, while facilitating safe and lawful deployment in clinical settings.

### 7.2. Function

The Regulatory and Compliance Layer is responsible for implementing and maintaining compliance with healthcare regulations, ensuring that patient data is protected, AI-driven decisions are transparent, and the system operates within the legal boundaries set by industry standards and government regulations.

### 7.3. Components

The Regulatory and Compliance Layer includes several critical components:

### 7.3.1. Data Privacy

### 7.3.1.1. HIPAA Compliance

The system strictly adheres to HIPAA standards for handling Protected Health Information (PHI), implementing stringent access controls, multi-factor authentication, and data anonymization to ensure that patient information remains confidential and secure.

### 7.3.1.2. Patient Consent Management

Mechanisms for obtaining and tracking patient consent are embedded within the system, ensuring that data usage is fully transparent and compliant with legal requirements. Patients can modify their consent preferences at any time, maintaining control over their personal health information.

### 7.3.2. Encryption

### 7.3.2.1. Data Encryption Standards

The system employs industry-leading encryption protocols (e.g., AES-256) for securing data both at rest and in transit, ensuring that sensitive information is protected from unauthorized access.

### 7.3.2.2. Data Integrity

The system uses cryptographic hashing algorithms and digital signatures to maintain the integrity and authenticity of data, ensuring that any unauthorized alterations are immediately detected.

### 7.3.3. Audit Trails

### 7.3.3.1. Comprehensive Logging

All significant events, including data access, AI-driven decisions, and clinician interactions, are logged in an immutable format, ensuring that the system maintains a detailed, tamper-proof audit trail for regulatory reviews and investigations.

### 7.3.4. Transparency and Accountability

### 7.3.4.1. Explainable AI (XAI)

The system incorporates Explainable AI (XAI) techniques to provide clear, understandable reasoning for all AI-driven decisions, with access to detailed decision logs available to authorized users, ensuring transparency and building trust. This includes providing clear, human-readable explanations for why certain predictions or recommendations were made, helping clinicians trust and verify the system's outputs.

### 7.3.4.2. Access to Decision Logs

Authorized users can access detailed logs of AI-driven decisions, allowing them to review the reasoning behind each recommendation. This transparency is crucial for building trust in the system and ensuring compliance with regulatory standards.

### 7.3.5. Regulatory Reporting

Automated reports summarizing compliance metrics, such as data access patterns and encryption status, can be generated for internal reviews or submission to regulatory bodies. Continuous compliance monitoring ensures that the system remains aligned with the latest regulatory standards.

### 7.3.5.1. Automated Compliance Reports

The system can generate automated reports that summarize key compliance metrics, such as data access patterns, encryption status, and audit log integrity. These reports can be used for internal reviews or submitted to regulatory bodies as required.

### 7.3.5.2. Continuous Compliance Monitoring

The system continuously monitors its operations to ensure ongoing compliance with relevant regulations and standards. If any issues are detected, such as unauthorized access attempts or deviations from standard protocols, the system generates alerts and provides recommendations for corrective actions.

### 7.4. Detailed Explanation

The Regulatory and Compliance Layer is integral to the system's ability to operate safely and legally within clinical environments. This layer addresses all aspects of data privacy, security, and regulatory compliance, ensuring that patient information is handled with the utmost care and transparency. The system's strict adherence to HIPAA standards, robust encryption protocols, and comprehensive audit trails guarantee that all data interactions are secure, traceable, and compliant with legal requirements. The inclusion of Explainable AI ensures that clinicians and regulators can understand and trust the system's recommendations, while automated reporting and continuous monitoring provide ongoing assurance that the system remains compliant with evolving regulations. This layer not only protects patient data but also builds confidence in the system's use in real-world clinical settings.

### 7.4.1. Data Privacy

### 7.4.1.1. HIPAA Compliance

### 7.4.1.1.1. Protected Health Information (PHI) Handling

The system is built to handle Protected Health Information (PHI) in strict accordance with the Health Insurance Portability and Accountability Act (HIPAA). This includes implementing safeguards for the confidentiality, integrity, and availability of PHI.

### 7.4.1.1.2. Access Controls

Role-Based Access Control (RBAC) mechanisms are in place to ensure that only authorized personnel have access to sensitive patient data. The system requires multi-factor authentication (MFA) for accessing PHI, adding an extra layer of security.

### 7.4.1.1.3. Data Anonymization

To further protect patient privacy, the system anonymizes data where possible, particularly in scenarios involving data analysis and sharing. This process removes personal identifiers, ensuring that data cannot be traced back to individual patients.

### 7.4.1.2. Patient Consent Management

### 7.4.1.2.1. Informed Consent

The system includes mechanisms for managing patient consent, ensuring that patients are fully informed about how their data will be used. This includes obtaining explicit consent before collecting or processing data for research or analysis purposes.

### 7.4.1.2.2. Consent Tracking

The system tracks consent decisions and allows patients to modify their consent preferences at any time. This ensures that the system always operates within the bounds of patient-approved data use.

### 7.4.2. Encryption

### 7.4.2.1. Data Encryption Standards

### 7.4.2.1.1. End-to-End Encryption

The system employs industry-standard encryption protocols (e.g., AES-256) to protect data both at rest and in transit. This ensures that patient data is secure from the moment it is collected to the point it is accessed or shared.

### 7.4.2.1.2. Key Management

Secure key management practices are in place to handle encryption keys. These keys are stored in secure environments, such as Hardware Security Modules (HSMs), and are regularly rotated to minimize the risk of unauthorized access.

### 7.4.2.2. Data Integrity

### 7.4.2.2.1. Hashing Algorithms

Cryptographic hashing algorithms (e.g., SHA-256) are used to ensure data integrity. This means that any unauthorized changes to the data will be immediately detectable, preventing tampering or corruption.

### 7.4.2.2.2. Digital Signatures

The system uses digital signatures to verify the authenticity of data and transactions. This provides a clear audit trail and ensures that all data accessed or modified within the system is traceable to an authorized source.

### 7.4.3. Audit Trails

### 7.4.3.1. Comprehensive Logging

### 7.4.3.1.1. Event Logging

The system logs all significant events, including data access, AI-driven decisions, and user interactions. Each log entry includes a timestamp, user ID, and detailed event description, providing a transparent record of system activity.

### 7.4.3.1.2. Immutable Logs

Logs are stored in an immutable format, meaning they cannot be altered or deleted once recorded. This ensures the integrity and reliability of the audit trail, making it a trustworthy source for compliance reviews and investigations.

### 7.4.3.2. Transparency and Accountability

### 7.4.3.2.1. Explainable AI

The system incorporates Explainable AI (XAI) techniques to ensure that all AI-driven decisions are transparent and understandable. This includes providing clear, human-readable explanations for why certain predictions or recommendations were made.

### 7.4.3.2.2. Access to Decision Logs

Authorized users have access to detailed logs of AI-driven decisions, allowing them to review the reasoning behind each recommendation. This transparency is crucial for building trust in the system and ensuring compliance with regulatory standards.

### 7.4.3.3. Regulatory Reporting

### 7.4.3.3.1. Automated Compliance Reports

The system can generate automated reports that summarize key compliance metrics, such as data access patterns, encryption status, and audit log integrity. These reports can be used for internal reviews or submitted to regulatory bodies as required.

### 7.4.3.3.2. Continuous Compliance Monitoring

The system continuously monitors for compliance with relevant regulations and standards. If any issues are detected, such as unauthorized access attempts or deviations from standard protocols, the system generates alerts and provides recommendations for corrective actions.

### 7.4.4. Regulatory and Compliance Layer Overview

This layer ensures that the AI-driven system operates within legal and ethical boundaries, adhering to all relevant healthcare regulations, including data privacy, security, and transparency standards such as HIPAA.

7.4.5. Reinforcement to Compete with competitors such as Watson

7.4.5.1. Enhanced Regulatory Focus

To ensure that this system outperforms platforms like IBM Watson, the patent emphasizes the system's deep integration with regulatory compliance, making it more suitable for the highly regulated field of T cell therapies.

### 7.4.5.2. Advanced Encryption and Privacy Protocols

Compared to Watson, this system could be positioned as offering superior data protection measures, particularly in the context of sensitive medical data related to engineered T cell therapies.

### 7.4.5.3. Explainable AI as a Core Feature

While Watson provides insights, this system's focus on Explainable AI (XAI) gives it an edge by ensuring that all decisions can be easily understood and justified, a critical factor in clinical environments where trust in AI decisions is paramount.

### 8. Integration and Workflow Layer

The Integration and Workflow Layer is the final component of the AI-driven system, designed to ensure that the system integrates smoothly with existing clinical workflows. This layer handles the practical aspects of implementing the system in real-world clinical settings, ensuring that data acquisition, analysis, and decision-making processes are streamlined and non-disruptive. By facilitating efficient interaction with other healthcare technologies and supporting the day-to-day activities of clinicians, this layer plays a vital role in the system's overall effectiveness.

### 8.1. Objective

To ensure that the AI-driven system integrates seamlessly into existing clinical workflows, supporting efficient data acquisition, analysis, and decision-making processes without disrupting current practices, thereby enhancing the overall efficiency and effectiveness of patient care.

### 8.2. Function

The Integration and Workflow Layer is responsible for facilitating the smooth deployment of the system within clinical environments, ensuring that it operates harmoniously with existing healthcare technologies, supports seamless data flow, and enhances clinician productivity through automation and real-time decision support.

8.3.

### Components

This layer includes several essential components to ensure effective integration:

### 8.3.1. Bidirectional Data Exchange

The system integrates with Electronic Health Records (EHR) and other healthcare information systems, enabling the seamless exchange of data in real time. This bidirectional flow ensures that patient data is up-to-date across all platforms, reducing the risk of errors and omissions.

### 8.3.2. Role-Based Dashboards

The system provides customized dashboards tailored to the needs of different members of the care team, such as oncologists, nurses, and pharmacists. These role-based dashboards ensure that each user has access to the most relevant data and tools for their specific role, improving efficiency and decision-making.

### 8.3.3. Automated Documentation

The system automates many of the routine documentation tasks, such as updating patient records with AI-driven recommendations, decisions, and outcomes. This reduces the administrative burden on clinicians, allowing them to focus more on patient care and less on paperwork.

### 8.4. Detailed Explanation

The Integration and Workflow Layer is designed to ensure that the AI-driven system can be implemented in clinical settings without disrupting existing workflows. By integrating seamlessly with Electronic Health Records (EHR) and other healthcare information systems, the system ensures that data flows smoothly between platforms, providing clinicians with up-to-date, accurate information at all times. The role-based dashboards offer tailored views and tools for different members of the care team, enabling them to access the most relevant data quickly and efficiently. Automation of documentation processes further enhances clinician productivity by reducing the time spent on administrative tasks, allowing more time for direct patient care. This layer ensures that the system not only fits into existing workflows but also enhances them, making the delivery of care more efficient and effective.

### 8.5. Integration and Workflow Layer Overview:

8.5.1. This layer ensures that the AI-driven system integrates seamlessly into existing clinical workflows. It handles the practical aspects of implementing the system in a real-world clinical setting, ensuring that data acquisition, analysis, and decision-making processes are streamlined and non-disruptive to healthcare providers.

8.6.
Seamless Integration with Clinical Systems

### 8.6.1. Electronic Health Record (EHR) Integration

The system is designed to integrate with hospital Electronic Health Records (EHR) systems, enabling bidirectional data exchange. Patient data from the EHR is automatically imported into the AI system for analysis, while AI-generated recommendations, decisions, and alerts are immediately updated in the patient's medical record.

### 8.6.2. Streamlined Documentation

The system automates much of the documentation process, reducing the administrative burden on clinicians. For example, when an alert is generated and a recommendation is followed, the system automatically documents the action and updates the patient's medical record, ensuring that all clinical decisions are accurately recorded.

### 8.7. Real-Time Monitoring and Decision Support

### 8.7.1. Continuous Data Flow

The system integrates with existing patient monitoring devices and systems, ensuring a continuous flow of real-time data. This includes interfacing with vital signs monitors, laboratory information systems (LIS), and imaging modalities, ensuring that all relevant patient data is available for AI-driven analysis.

### 8.7.2. Real-Time Alerts and Recommendations

The system continuously analyzes the incoming data and provides real-time alerts and therapeutic recommendations. These are displayed on an interactive dashboard, allowing clinicians to make informed decisions quickly and efficiently.

### 8.8. Customization and Personalization

### 8.8.1. Role-Based Dashboards

The system offers customized dashboards tailored to the specific roles of different healthcare providers. For example, oncologists might prioritize cytokine trends and immune cell counts, while nurses might focus on vital signs and patient-reported symptoms. This personalization ensures that each team member has access to the most relevant information for their role.

### 8.8.2. Customizable Protocols

Clinicians can customize the system's monitoring and alert thresholds based on the patient's condition and treatment goals. This allows for a personalized monitoring strategy that adapts to the specific needs of each patient.

### 8.9. Interdisciplinary Collaboration

### 8.9.1. Collaboration Tools

The system includes tools that facilitate communication and collaboration among the care team. Clinicians can share notes, discuss alerts, and coordinate interventions directly within the system, ensuring that everyone involved in the patient's care is on the same page.

### 8.9.2. Decision Logging and Feedback

Every decision made using the system's recommendations is logged, including the data that informed the decision and the outcome. This logging ensures a clear record of how clinical decisions were made, which is valuable for both compliance and quality improvement.

### 8.10. Patient Monitoring

8.10.1.continuous Data Flow

8.10.1.1. Real-Time Data Integration

The system integrates with existing patient monitoring devices and systems, ensuring a continuous flow of real-time data. This includes interfacing with vital signs monitors, laboratory information systems (LIS), and electronic health records (EHR).

### 8.10.1.2. Data Synchronization

The system synchronizes data from multiple sources, such as lab results and imaging studies, to provide a comprehensive view of the patient's condition. This ensures that all relevant data is available in one place, reducing the need for clinicians to switch between systems.

### 8.10.2.Cytokine and Immune Monitoring

### 8.10.2.1. Real-Time Cytokine Monitoring

The system continuously monitors cytokine levels, dendritic cell activity, and macrophage function. This data is analyzed in real-time to detect early signs of adverse immune reactions, such as Cytokine Release Syndrome (CRS).

### 8.10.2.2. Adaptive Monitoring Protocols

The system adapts its monitoring protocols based on the patient's condition and response to therapy. For example, if cytokine levels begin to rise, the system may increase the frequency of data collection or focus on specific biomarkers.

### 8.10.3.Therapeutic Decision-Making

### 8.10.3.1. Real-Time Alerts and Suggestions

### 8.10.3.1.1. Immediate Intervention Recommendations

When the system detects a significant change in the patient's condition, it immediately generates alerts and therapeutic suggestions. For instance, if the system predicts a high risk of CRS, it may recommend administering a cytokine inhibitor or adjusting the T cell infusion rate.

### 8.10.3.1.2. Customizable Alert Thresholds

Clinicians can customize alert thresholds based on the patient's baseline levels and treatment goals. This personalization ensures that alerts are meaningful and actionable, reducing the likelihood of alarm fatigue.

### 8.10.3.2. Decision Logging and Outcome Feedback

### 8.10.3.2.1. Decision Logging

Every decision made using the system's recommendations is logged, including the data that informed the decision and the outcome. This logging ensures that there is a clear record of how clinical decisions were made, which is valuable for both compliance and quality improvement.

### 8.10.3.2.2. Outcome Feedback Loop

The system uses outcome data to refine its predictive models and decision support tools. For example, if a recommended intervention successfully prevents an adverse event, the system will adjust its algorithms to prioritize similar recommendations in the future.

### 8.10.4. Seamless Workflow Integration

### 8.10.4.1. EHR Integration

### 8.10.4.1.1. Bidirectional Data Exchange

The system integrates with the hospital's EHR system, allowing for bidirectional data exchange. This means that patient data from the EHR is automatically available to the AI system, and any new data or decisions made by the system are immediately updated in the EHR.

### 8.10.4.1.2. Streamlined Documentation

The system automates much of the documentation process, reducing the administrative burden on clinicians. For example, when an alert is generated and a recommendation is followed, the system automatically documents the action and updates the patient's medical record.

### 8.10.4.2. Interdisciplinary Collaboration

### 8.10.4.2.1. Collaboration Tools

The system includes tools that facilitate communication and collaboration among the care team. Clinicians can share notes, discuss alerts, and coordinate interventions directly within the system, ensuring that everyone is on the same page.

### 8.10.4.2.2. Role-Based Dashboards

Different members of the care team can access customized dashboards tailored to their specific roles. For example, oncologists might focus on immune monitoring and cytokine levels, while nurses might prioritize vital signs and patient-reported symptoms.

### 9. Innovative Features

### 9.1. Real-Time Data Integration

The system's ability to integrate real-time data from multiple sources, including vital signs, laboratory results, and imaging, sets it apart from existing solutions that rely on periodic or retrospective data analysis.

### 9.2. Predictive Precision

The use of advanced AI techniques, such as LSTM networks, enables the system to detect subtle trends and interactions between different physiological parameters, providing more accurate and timely predictions of adverse events.

### 9.3. Adaptive Clinical Decision Support

By continuously learning from patient outcomes, the system tailors its recommendations to the specific needs of each patient, optimizing therapy on an individual basis.

### REFERENCES

Hayday A C. γδ T cells and the lymphoid stress-surveillance response. Immunity. 2009;31(2):184-196. doi:10.1016/j.immuni.2009.08.006.
Makkouk A, Weiner G J. Cancer Immunotherapy and Breaking Immune Tolerance: New Approaches to an Old Challenge. Cancer Res. 2015;75(1):5-10. doi:10.1158/0008-5472.CAN-14-1871.
Groh V, et al. Stimulation of CD94/NKG2C by Endogenous NKG2C Ligands Confers Enhanced Effector Functions to Human NK Cells in Tumor Microenvironments. Proc Natl Acad Sci U S A. 2013;110(24):9521-9526. doi:10.1073/pnas.1306248110.
Bauer S, Groh V, Wu J, et al. Activation of NK Cells and T Cells by NKG2D, a Receptor for Stress-Inducible MICA. Science. 1999;285(5428):727-729. doi:10.1126/science.285.5428.727.
Cheng M, et al. Targeting Antibody-Dependent Cellular Cytotoxicity (ADCC) in Tumor Therapy. Antib Ther. 2017;12(6):391-402. doi:10.1080/2162402X.2016.1271948.
Watanabe S, et al. Antibody-dependent cellular cytotoxicity mediated by γδ T cells: implications for cancer immunotherapy. Cancer Immunol Res. 2019;7(2):145-151. doi:10.1158/2326-6066.CIR-18-0347.
Lo Presti E, et al. γδ T cells and their versatility: How they orchestrate innate and adaptive immunity against infections and cancer. Front Immunol. 2014;5:407. doi:10.3389/fimmu.2014.00407.
Wu Y, Tian Z, Wei H. Developmental and Functional Control of Natural Killer Cells by Cytokines. Front Immunol. 2017;8:930. doi:10.3389/fimmu.2017.00930.
Fujisawa T, et al. γδ T cells in cancer: a double-edged sword. Cancer Immunol Res. 2016;4(8):684-694. doi:10.1158/2326-6066.CIR-15-0271.
Maaten J V, et al. Cytokine secretion by γδ T cells in the tumor microenvironment: implications for anti-tumor immunity. Cytokine Growth Factor Rev. 2020;55:73-81. doi:10.1016/j.cytogfr.2020.06.002.
Dieli F, et al. γδ T cells modulate the anti-cancer immune response and promote angiogenesis in human pancreatic cancer. J Immunol. 2014;192(2):1016-1023. doi:10.4049/jimmunol.1302027.
Kroemer A, et al. γδ T cells and their potential roles in regulating angiogenesis. Cell Mol Immunol. 2017;14(5):419-428. doi:10.1038/cmi.2017.4.
Vantourout P, Hayday A. Six-of-the-best: unique contributions of γδ T cells to immunology. Nat Rev Immunol. 2013;13(2):88-100. doi:10.1038/nri3384.
Dimova T, et al. Effector γδ T cells and tissue residency: the interactions and modulation by immune checkpoints. Cancer Immunol Res. 2020;8(9):1201-1209. doi:10.1158/2326-6066.CIR-20-0052.
Zhao Y, et al. γδ T cells: Diverse functions in autoimmunity and inflammation. Cell Mol Immunol. 2018;15(5):416-426. doi: 10.1038/s41423-018-0039-z.
Paul S, et al. Cytokine regulation of γδ T cells in autoimmune diseases. Immunology. 2020;160(1):5-15. doi:10.1111/imm.13192.
Brandes M, Willimann K, Moser B. Professional antigen-presentation function by human γδ T Cells. Science. 2005;309(5732):264-268. doi:10.1126/science.1110267.
Xie Y, et al. γδ T cells in immune regulation: Focus on their role in regulating Treg cells. Immunol Rev. 2020;298(1):48-59. doi:10.1111/imr.12921.
Lanzavecchia A, Sallusto F. Dynamics of T lymphocyte responses: intermediates, effectors, and memory cells. Science. 2000;290(5489):92-97. doi:10.1126/science.290.5489.92.
Hayday A C. γδ T cell update: adapting innate-like immunity to fight infection. Nat Rev Immunol. 2019;19(7):463-478. doi:10.1038/s41577-019-0167-7.
Bonneville M, O'Brien R L, Born W K. γδ T cell effector functions: a blend of innate programming and acquired plasticity. Nat Rev Immunol. 2010;10(7):467-478. doi:10.1038/nri2781.
Poccia F, Agrati C, Martini F, et al. Antiviral reactivities of γδ T cells. Microbes Infect. 2005;7(3):518-528. doi:10.1016/j. micinf.2004.12.015.
Koehler A B, et al. Chemokines and adhesion molecules orchestrate the migration and localization of γδ T cells to tumor sites. Cancer Immunol Immunother. 2016;65(12):1467-1479. doi:10.1007/s00262-016-1887-4.
Moser B, Eberl M. γδ T cells: novel initiators of adaptive immunity. Immunol Rev. 2011;240(1):40-51. doi:10.1111/j.1600-065X.2010.00999.x.
Wesch D, Peters C, Oberg HH, et al. Mechanisms of γδ T cell-mediated cytotoxicity against tumor cells. Front Immunol. 2014; 5:499. doi: 10.3389/fimmu.2014.00499.
Groh V, et al. Stress-induced recognition by γδ T cells as a target for cancer immunotherapy. Cell. 1998;95(7):1011-1020. doi:10.1016/S0092-8674(00)81725-1.
Brandes M, Willimann K, Moser B. Professional antigen-presentation function by human γδ T Cells. Science. 2005;309(5732):264-268. doi:10.1126/science.1110267.
Wu Y, et al. γδ T cells: Functions in autoimmune diseases and their potential as a therapeutic target. Front Immunol. 2021;12:700987. doi:10.3389/fimmu.2021.700987.
Paul S, et al. Cytokine regulation of γδ T cells in autoimmune diseases. Immunology. 2020;160(1):5-15. doi:10.1111/imm.13192.
Bonneville M, et al. γδ T cells in cancer: another look at the outcome. Nat Rev Immunol. 2010;10(7):460-471. doi:10.1038/nri2780.
Buccheri S, et al. γδ T cells in cancer treatment: The next wave of immunotherapy. Front Immunol. 2014;5:678. doi:10.3389/fimmu.2014.00678.
Hayday A C. γδ T cell update: Adapting innate-like immunity to fight infection. Nat Rev Immunol. 2019;19(7):463-478. doi:10.1038/s41577-019-0167-7.
Dieli F, et al. γδ T cells modulate the anti-cancer immune response and promote angiogenesis in human pancreatic cancer. J Immunol. 2014;192(2):1016-1023. doi:10.4049/jimmunol.1302027.
Saito H, et al. γδ T cells and cancer. Curr Opin Immunol. 2021;67:75-83. doi:10.1016/j.coi.2020.12.005.
Girardi M, et al. Regulation of immune responses by γδ T cells. Immunol Rev. 2009;229(1):107-117. doi:10.1111/j.1600-065X.2009.00765.x.
Vantourout P, Hayday A. Six-of-the-best: unique contributions of γδ T cells to immunology. Nat Rev Immunol. 2013;13(2):88-100. doi:10.1038/nri3384.
Dieli F, et al. Regulation of γδ T cells in anti-tumor immunity. Nat Rev Immunol. 2001;1(6):491-498. doi:10.1038/35106159.
Hayday A. γδ T cells in cancer: protection and pathogenesis. Nat Rev Immunol. 2009;9(7):470-477. doi:10.1038/nri2567.
Peng G, et al. Anti-tumor activity of γδ T cells in cancer: Mechanisms and potential immunotherapy. Front Immunol. 2015;6:6. doi:10.3389/fimmu.2015.00006.
Wu P, et al. γδ T cells and tumor microenvironment: a multifaceted interplay. Front Immunol. 2020;11:969. doi:10.3389/fimmu.2020.00969.
Lo Presti E, et al. γδ T cells as a potential player in immunotherapy: Present and future perspectives. Front Immunol. 2017;8:1613. doi:10.3389/fimmu.2017.01613.
Gao Y, et al. γδ T cells in anti-tumor immunity: Present and future perspectives. Front Immunol. 2017;8:1613. doi:10.3389/fimmu.2017.01613.
Fisher J P, et al. Clinical monitoring of γδ T cell responses during cancer immunotherapy. Front Immunol. 2014;5:654. doi:10.3389/fimmu.2014.00654.
Wesch D, et al. Monitoring γδ T cell responses in cancer patients: Clinical implications and future perspectives. Cancer Immunol Immunother. 2014;63(6):579-589. doi:10.1007/s00262-014-1546-4.
Jackson H J, Rafiq S, Brentjens R J. Driving CAR T-cells forward. Nat Rev Clin Oncol. 2016;13(6):370-383. doi:10.1038/nrclinonc.2016.36.
Gross G, Waks T, Eshhar Z. Expression of immunoglobulin-T-cell receptor chimeric molecules as functional receptors with antibody-type specificity. Proc Natl Acad Sci U S A. 1989;86(24):10024-10028. doi:10.1073/pnas.86.24.10024.
Fesnak A D, June C H, Levine B L. Engineered T cells: the promise and challenges of cancer immunotherapy. Nat Rev Cancer. 2016;16(9):566-581. doi:10.1038/nrc.2016.97.
Finney H M, Lawson A D, Bebbington C R, Weir A N. Chimeric receptors providing both primary and costimulatory signaling in T cells from a single gene product. J Immunol. 1998;161(6):2791-2797.
Kalos M, Levine B L, Porter D L, et al. T cells with chimeric antigen receptors have potent antitumor effects and can establish memory in patients with advanced leukemia. Sci Transl Med. 2011;3(95):95ra73. doi:10.1126/scitranslmed.3002842.
Zheng P P, Kros J M, Li J. Approved CAR T cell therapies: ice bucket challenges on glaring safety risks and long-term impacts. Drug Discov Today. 2018;23(6):1175-1182. doi:10.1016/j. drudis.2018.03.007.
Koneru M, Purdon T J, Spriggs D, Koneru S, Brentjens R J. IL-12 secreting tumor-targeted chimeric antigen receptor T cells eradicate ovarian tumors in vivo. Oncoimmunology. 2015;4(3). doi:10.1080/2162402X.2014.994446.
Martinez M, Moon E K. CAR T cells for solid tumors: new strategies for finding, infiltrating, and surviving in the tumor microenvironment. Front Immunol. 2019;10:128. doi:10.3389/fimmu.2019.00128.
Xu Y, Zhang M, Ramos C A, et al. Closely related T-memory stem cells correlate with in vivo expansion of CAR. Blood. 2014;123(24):3750-3759. doi:10.1182/blood-2014-01-552174.
Brentjens R J, Davila M L, Riviere I, et al. CD19-targeted T cells rapidly induce molecular remissions in adults with chemotherapy-refractory acute lymphoblastic leukemia. Sci Transl Med. 2013;5(177):177ra38. doi:10.1126/scitranslmed.3005930.
Gattinoni L, Klebanoff C A, Restifo N P. Paths to stemness: building the ultimate antitumor T cell. Nat Rev Cancer. 2012;12(10):671-684. doi:10.1038/nrc3322.
Turtle C J, Hanafi L A, Berger C, et al. CD19 CAR-T cells of defined CD4++ composition in adult B cell ALL patients. J Clin Invest. 2016;126(6):2123-2138. doi:10.1172/JCI85309.
Restifo N P, Dudley M E, Rosenberg S A. Adoptive immunotherapy for cancer: harnessing the T cell response. Nat Rev Immunol. 2012;12(4):269-281. doi:10.1038/nri3191.
Wherry E J. T cell exhaustion. Nat Immunol. 2011;12(6):492-499. doi:10.1038/ni.2035.
Pegram H J, Lee J C, Hayman E G, et al. Tumor-targeted T cells modified to secrete IL-12 eradicate systemic tumors without need for prior conditioning. Blood. 2012;119(18):4133-4141. doi:10.1182/blood-2011-10-387365.
Porter D L, Levine B L, Kalos M, Bagg A, June C H. Chimeric antigen receptor-modified T cells in chronic lymphoid leukemia. N Engl J Med. 2011;365(8):725-733. doi:10.1056/NEJMoa1103849.
Newick K, O'Brien S, Moon E, Albelda S M. CAR T cell therapy for solid tumors. Annu Rev Med. 2017;68:139-152. doi:10.1146/annurev-med-062315-120245.
Beatty G L, O'Hara M. Chimeric antigen receptor-modified T cells: successes and challenges in cancer immunotherapy. Cancer J. 2016;22(2):73-82. doi:10.1097/PPO.
Schuster S J, Svoboda J, Chong E A, et al. Chimeric antigen receptor T cells in refractory B-cell lymphomas. N Engl J Med. 2017;377(26):2545-2554. doi:10.1056/NEJMoa1708566.
Sotillo E, Barrett D M, Black K L, et al. Convergence of acquired mutations and alternative splicing of CD19 enables resistance to CART-19 immunotherapy. Cancer Discov. 2015; 5(12):1282-1295. doi:10.1158/2159-8290.CD-15-1020.
Jiang Y, Li Y, Zhu B. T-cell exhaustion in the tumor microenvironment. Cell Death Dis. 2015;6. doi:10.1038/cddis.2015.162.
Wherry E J, Kurachi M. Molecular and cellular insights into T cell exhaustion. Nat Rev Immunol. 2015;15(8):486-499. doi:10.1038/nri3862.
Zhang Q, Wang H, Kornepati A V, et al. The role of TGF-β in cancer immunotherapy: TGF-β blockade in CAR T-cell therapy and beyond. J Hematol Oncol. 2021;14(1):153. doi:10.1186/s13045-021-01149-5.
Loo Yau H, Bell E, Ettayebi I, et al. DNA hypomethylating agent enhances anti-tumor activity of T cells with chimeric antigen receptor by downregulating immunosuppressive genes. J Immunother Cancer. 2021;9(3).

1. An AI-driven data analytics system for optimizing engineered T cell therapies in cancer, autoimmune, and inflammatory conditions, comprising a data acquisition module for collecting and integrating real-time data from monitoring systems, laboratory tests, imaging studies, wearable devices, immune profiling, and environmental data, a preprocessing module for ensuring high-fidelity data using noise reduction, normalization, real-time data imputation, and dynamic filtering, a hybrid AI model combining techniques such as Long Short-Term Memory (LSTM) networks, Random Forests, Convolutional Neural Networks (CNNs), and fuzzy logic algorithms to predict adverse events like Cytokine Release Syndrome and Tumor Lysis Syndrome, a monitoring and alerting module to generate real-time alerts and adjust monitoring parameters based on treatment phase and patient condition, a decision support module for providing personalized guidance to clinicians by integrating AI-driven predictive analytics with real-time patient data and clinical guidelines, and a feedback module that incorporates reinforcement learning to refine AI algorithms based on clinical outcomes, post-market surveillance, and real-world evidence.

2. The system of claim 1, wherein the data acquisition module prioritizes critical parameters for real-time monitoring post-infusion of T cell therapies, including cytokine levels (IL-6, TNF-α, IFN-γ), electrolyte levels (potassium, uric acid, phosphorus, calcium), vital signs (heart rate, blood pressure, respiratory rate, oxygen saturation), renal function markers (serum creatinine, eGFR, urine output), cardiac function markers (ECG parameters, cardiac enzymes), and neurological function (EEG data, neurological assessments) to ensure comprehensive patient monitoring during and after therapy.

3. The system of claim 1, wherein the AI algorithm module integrates multi-modal data, including genetic data, imaging studies, patient-reported outcomes, and real-time biomarker levels, to enhance predictive accuracy and enable proactive management of adverse events in CAR T cell therapy, Gamma Delta T cell therapy, dendritic cell therapy, and NK cell therapy.

4. The system of claim 1, wherein the monitoring and alerting module adjusts the prioritization of monitoring parameters based on real-time clinical data during CAR T cell therapy, Gamma Delta T cell therapy, dendritic cell therapy, and NK cell therapy, ensuring relevant data points are emphasized as patient conditions evolve, and providing clinicians with actionable insights.

5. The system of claim 1, wherein the decision support module provides comparative analytics across CAR T, Gamma Delta T, dendritic cell, and NK cell therapies, incorporating evidence-based algorithms for managing CRS and TLS, allowing clinicians to make data-driven decisions based on the effectiveness, risks, and patient-specific profiles of each therapy.

6. The system of claim 1, wherein the AI-driven predictive models are specifically trained on datasets that include historical data from T cell therapies, ensuring that predictions are finely tuned to the unique physiological responses associated with CAR T cell therapy, Gamma Delta T cell therapy, dendritic cell therapy, and NK cell therapy, thereby optimizing therapeutic strategies and reducing the risk of adverse events.

7. The system of claim 1, wherein the feedback and learning module incorporates data from ongoing clinical trials, post-market surveillance, real-world evidence, and clinical practice, enhancing the system's predictive accuracy, the personalization of therapeutic recommendations, and the continuous improvement of AI algorithms over time.

8. The system of claim 1, wherein the data acquisition module supports real-time integration of immune profiling data, including T cell receptor (TCR) sequencing, cytokine assays, and other immunological markers, which are critical for assessing the efficacy, safety, and personalized optimization of CAR T cell therapy, Gamma Delta T cell therapy, dendritic cell therapy, and NK cell therapy.

9. The system of claim 1, wherein the decision support module includes predictive analytics feature that forecasts potential adverse events such as CRS and TLS based on multi-dimensional data inputs, enabling preemptive interventions, personalized treatment plans, and the dynamic adjustment of therapeutic protocols in real-time.

10. The system of claim 1, wherein the AI algorithm module includes specific sub-algorithms for managing multi-system interactions during T cell therapy, such as the interplay between immune responses, renal function, cardiac stability, and neurological function, predicting and preventing complex adverse events like multi-organ failure and neurotoxicity.

11. The system of claim 1, wherein the data preprocessing module employs machine learning algorithms to automatically prioritize and weight parameters that are most predictive of CRS, TLS, and other severe adverse events in real-time, enhancing the system's ability to predict and prevent complications through continuous analysis and adaptive learning.

12. The system of claim 1, wherein the decision support module integrates real-time clinical data with established clinical guidelines, patient-specific factors, and comparative analytics to provide dynamic, personalized treatment recommendations for the management of CRS, TLS, and other complications, including the adjustment of T cell therapy dosing, pharmacological interventions, and supportive care protocols.

13. The system of claim 1, wherein the monitoring and alerting module includes an escalation protocol that automatically triggers more intensive monitoring, intervention measures, and multidisciplinary team involvement when the AI algorithms detect a high likelihood of severe CRS, TLS, or other critical events, ensuring early and decisive action to prevent life-threatening complications.

14. The system of claim 1, wherein the feedback and learning module continuously refines the AI algorithms'predictive accuracy and therapeutic recommendations based on real-world evidence, including data from clinical practice, post-market surveillance, ongoing clinical trials, and patient outcomes, thereby enhancing the system's ability to adapt to evolving clinical practices and patient populations.

15. The system of claim 1, wherein the data acquisition module integrates external health information systems, clinical databases, and population health statistics to access historical patient data, relevant datasets, and broader epidemiological trends, enhancing the predictive capabilities of the AI algorithms for managing CRS, TLS, and other adverse events in T cell therapies.

16. The system of claim 1, wherein the decision support module incorporates comparative analytics to evaluate the effectiveness, risks, and potential synergies of different T cell therapies (CAR T, Gamma Delta T, dendritic cell, NK cell), providing clinicians with insights into the comparative benefits, trade-offs, and optimal therapeutic strategies for each patient based on real-time and historical data.

17. The system of claim 1, wherein the AI algorithm module is configured to dynamically adjust its predictive models based on real-time data inputs, continuously refining risk assessments, therapeutic recommendations, and monitoring protocols to adapt to the unique physiological responses and evolving clinical conditions of each patient undergoing T cell therapy.

18. The system of claim 1, wherein the monitoring and alerting module includes a visualization dashboard that allows clinicians to interactively explore patient data, risk scores, predictive analytics, and historical trends, facilitating informed, data-driven decision-making in real-time for the management of CRS, TLS, and other complications during T cell therapy.

19. The system of claim 1, wherein the feedback and learning module logs all AI-driven recommendations, clinician actions, patient outcomes, and system adjustments, creating a comprehensive audit trail that is used for ongoing model refinement, regulatory compliance, and quality assurance, ensuring transparency, accountability, and continuous improvement in clinical decision-making.

20. The system of claim 1, wherein the data acquisition module specifically includes the integration of immune profiling data, such as T cell receptor (TCR) sequencing and cytokine assays, which are critical for assessing the efficacy, safety, and personalized optimization of CAR T cell therapy, Gamma Delta T cell therapy, dendritic cell therapy, and NK cell therapy, thereby enhancing the system's ability to predict, prevent, and manage adverse events through precise, data-driven interventions.

21. A method for managing side effects in engineered T cell therapies, comprising continuous real-time analysis of patient data to detect potential side effects before they become clinically significant, delivering actionable, AI-driven recommendations to clinicians for the immediate implementation of therapeutic interventions, including the adjustment of treatment protocols, administration of rescue medications, intensification of monitoring efforts, and deployment of multidisciplinary care resources to prevent the escalation of adverse events, incorporating clinician feedback and post-event analysis to refine predictive models, improve accuracy and effectiveness of side effect management protocols, and enhance patient safety and therapeutic outcomes over time.
